(19) United States
(12) Patent Application Publication
(10) Pub. No.: US 2019/0133519 A1
(43) Pub. Date: May 9, 2019

(54) MONITORING TISSUE TREATMENT USING THERMOGRAPHY

(71) Applicants: Ramot at Tel-Aviv University Ltd., Tel-Aviv (IL); Tel HaShomer Medical Research Infrastructure and Services Ltd., Ramat-Gan (IL); Afeka Yissumim Ltd., Tel-Aviv (IL)

(72) Inventors: Israel GANNOT, Ramat-HaSharon (IL); Oshrit HOFFER, Kiryat-Ono (IL); Dror ALEZRA, Rishon-LeZion (IL); Merav A. BEN-DAVID, Kiryat-Ono (IL); Eyal KATZ, Ramat-Gan (IL)

(73) Assignees: Ramot at Tel-Aviv University Ltd., Tel-Aviv (IL); Tel HaShomer Medical Research Infrastructure and Services Ltd., Ramat-Gan (IL); Afeka Yissumim Ltd., Tel-Aviv (IL)

(21) Appl. No.: 16/233,361

(22) Filed: Dec. 27, 2018

Related U.S. Application Data
(63) Continuation of application No. PCT/IL2017/050717, filed on Jun. 27, 2017.
(60) Provisional application No. 62/354,905, filed on Jun. 27, 2016.

(51) Int. Cl. A61B 5/00; G06T 7/00; A61B 5/01; A61N 5/10

(52) U.S. Cl. CPC A61B 5/4848; G06T 7/0016; A61B 5/015; A61B 5/0091; H04N 5/33; A61B 5/0086; G06T 2207/30096; G06T 2207/30101; G06T 2207/10048; A61N 5/1001

(57) ABSTRACT

A method of monitoring a malignant tissue response to cancer treatment, including:
acquiring, throughout a treatment course, one or more thermal images of the treated malignant tissue;
processing the one or more thermal images to detect changes in the malignant tissue following the treatment; and
analyzing the processed images to determine an effect of the treatment on the malignant tissue based on said detected changes.

### RELATED APPLICATIONS

This application is a Continuation of PCT Patent Application No. PCT/IL2017/050717 having International filing date of Jun. 27, 2017, which claims the benefit of priority under 35 USC § 119(e) of U.S. Provisional Patent Application No. 62/354,905 filed on Jun. 27, 2016. The contents of the above applications are all incorporated by reference as if fully set forth herein in their entirety.

### FIELD AND BACKGROUND OF THE INVENTION

The present invention, in some embodiments thereof, relates to monitoring cancer treatment and, more particularly, but not exclusively, to use of thermography as a tool for assessing cancer treatment.

### SUMMARY OF THE INVENTION

According to an aspect of some embodiments of the invention, there is provided a method of monitoring a tissue response to cancer treatment, comprising: acquiring, throughout a treatment course, one or more thermal images of the treated tissue region; processing the one or more thermal images to detect tumor changes and vasculature; and analyzing the processed images to determine an effect of the treatment on the tissue based on the detected vasculature.

In some embodiments, the treated tissue region comprises malignant tissue.

In some embodiments, at least two thermal images are acquired and processing comprises comparing the thermal images to determine one or more changes in the tumor and in vasculature that are indicative of the tissue response to treatment.

In some embodiments, the processing comprises identifying one or more of narrow vessels, vessels with irregular curvature, and dense vasculature associated with the malignant tissue.

In some embodiments, the malignant tissue is a tumor and the detected vasculature comprises blood vessels and capillaries supplying blood to the tumor.

In some embodiments, changes in vasculature comprise one or more of a change in vessel curvature, a change in vessel diameter, and a change in vascular density.

In some embodiments, processing comprises distinguishing between temperatures caused by inflammation of the tissue, temperatures associated with a change in the tumor, and temperatures associated with vasculature.

In some embodiments, processing comprises applying one or more image processing algorithms configured to accentuate vasculature in the processed image.

In some embodiments, the algorithm is configured to accentuate malignant tissue in the processed image.

In some embodiments, the malignant tissue appears a bright spot in the processed image, and differences in size and/or brightness of the spot are indicative of differences in a size or malignancy level of the malignant tissue respectively.

In some embodiments, the algorithm is configured to normalize a temperature distribution of a target tissue region relative to a temperature distribution of a non-targeted tissue region that underwent the same the treatment.

In some embodiments, the algorithm is configured to mask effects of tissue heated due to inflammation.

In some embodiments, the algorithm takes into account tissue regions that are naturally warmer or colder than other tissue regions due to anatomy.

In some embodiments, the algorithm takes into account a geometry of the malignant tissue and/or a location of the malignant tissue relative to the skin surface.

In some embodiments, the cancer treatment comprises radiotherapy and/or chemotherapy and/or hormonal treatment.

In some embodiments, the acquiring is performed at a plurality of predetermined timings throughout the treatment course.

In some embodiments, timings are selected in accordance with a dose administered to the patient.

In some embodiments, processing comprises analyzing a condition of the vasculature to determine treatment-induced endothelial cell death in the malignant tissue.

In some embodiments, acquiring is performed externally to the patient's body.

In some embodiments, acquiring is performed internally to the patient's body.

In some embodiments, the acquiring is performed via a thermal camera mounted on an endoscope.

In some embodiments, the treated tissue region comprises breast tissue.

According to an aspect of some embodiments of the invention, there is provided a system for monitoring cancer treatment using thermography, comprising: a thermal imaging camera suitable for acquiring thermal images of a tissue region in which malignant tissue is present; a controller programmed to operate the camera one or more times throughout a treatment course according to one or more predefined protocols; and a processor configured to analyze the acquired thermal images for indicating the tissue response to treatment based on a condition of vasculature associated with the malignant tissue.

In some embodiments, the processor is programmed to apply one or more image processing algorithms designed to identify the vasculature condition or changes therein.

In some embodiments, the system is configured to provide a progress related indication for determining the efficacy of treatment.

In some embodiments, the system is configured to be integrated in and/or added onto an irradiating modality.

In some embodiments, the system is configured to automatically modify an irradiation scheme of the irradiating modality based on real time feedback obtained from the thermal images.

In some embodiments, the camera comprises an infrared resolution of at least 320×256 pixels.

According to an aspect of some embodiments of the invention, there is provided a device for personal follow-up post cancer treatment, comprising a thermal imaging camera suitable for acquiring thermal images of a treated tissue region; and a control module configured to control operation of the camera and to process the thermal images to provide an indication associated with malignant tissue previously treated by the treatment.

In some embodiments, the thermal imaging camera is configured to be integrated in and/or added on a smartphone, and wherein the control module comprises a smartphone application.

In some embodiments, the device is configured to provide an indication of recurrence of a previously treated condition.

According to an aspect of some embodiments of the invention, there is provided a method of determining tumor condition, comprising: acquiring one or more thermal images of a tissue region in which the tumor is found; processing the one or more thermal images to detect vasculature; and analyzing the processed images to determine a condition of the tumor based on the vasculature and tumor functional and structural changes.

In some embodiments, the condition comprises one or more of a size, volume, spread, and stage of the tumor.

### SOME EXAMPLES OF SOME EMBODIMENTS OF THE INVENTION ARE LISTED BELOW

Example 1. A method of monitoring a malignant tissue response to cancer treatment, comprising:
acquiring, throughout a treatment course, one or more thermal images of the treated malignant tissue;
processing said one or more thermal images to detect changes in said malignant tissue following said treatment; and
analyzing the processed images to determine an effect of said treatment on said malignant tissue based on said detected changes.
Example 2. The method according to example 1, wherein said malignant tissue comprises vasculature and/or tumor.
Example 3. The method according to example 2, wherein said vasculature are located outside said tumor and/or within said tumor.
Example 4. The method according to examples 1 or 2, further comprising delivering an indication to a user based if said effect is not a desired effect.
Example 5. The method according to example 2, wherein at least two thermal images are acquired and wherein said processing comprises comparing said at least two thermal images to determine one or more changes in said vasculature and/or said tumor that are indicative of a response of said malignant tissue to said treatment.
Example 6. The method according to example 1, wherein said processing comprises identifying one or more of vessel irregularities associated with the presence of a tumor in said malignant tissue.
Example 7. The method according to example 6, wherein said vessel irregularities comprise: narrow vessels, vessels with irregular curvature, and dense vasculature associated with said malignant tissue.
Example 8. The method according to example 2, wherein said detected vasculature comprises blood vessels supplying blood to said tumor.
Example 9. The method according to example 5, wherein said changes in vasculature comprise one or more of a change in vessel curvature, a change in vessel diameter, and a change in vascular density.
Example 10. The method according to example 2, wherein said processing comprises distinguishing between temperatures caused by inflammation of the tissue, temperatures associated with a change in the tumor, and temperatures associated with said vasculature.
Example 11. The method according to example 1, wherein said processing comprises applying one or more image processing algorithms configured to accentuate vasculature in the processed image.
Example 12. The method according to example 1, wherein said processing comprises applying one or more image processing algorithms configured to accentuate and detect vasculature in the processed image.
Example 13. The method according to example 11, wherein said algorithm is configured to accentuate said malignant tissue in the processed image.
Example 14. The method according to example 13, wherein said malignant tissue appears a bright spot in said processed image, and wherein differences in size and/or brightness of said spot are indicative of differences in a size or malignancy level of said malignant tissue respectively.
Example 15. The method according to example 11, wherein said algorithm is configured to normalize a temperature distribution of a target tissue region relative to a temperature distribution of a non-targeted tissue region that underwent the same the treatment.
Example 16. The method according to example 11, wherein said algorithm is configured to mask effects of tissue heated due to inflammation.
Example 17. The method according to example 11, wherein said algorithm takes into account tissue regions that are naturally warmer or colder than other tissue regions due to anatomy.
Example 18. The method according to example 11, wherein said algorithm takes into account a geometry of said malignant tissue and/or a location of said malignant tissue relative to the skin surface.
Example 19. The method according to example 1, wherein said cancer treatment comprises radiotherapy and/or brachytherapy and/or chemotherapy and/or immunotherapy and/or hormonal treatment.
Example 20. The method according to example 1, wherein said acquiring is performed at a plurality of predetermined timings throughout said treatment course.
Example 21. The method according to example 20, wherein said timings are selected in accordance with a dose administered to the patient.
Example 22. The method according to example 2, wherein said processing comprises analyzing a condition of said vasculature to determine treatment-induced endothelial cell death in said malignant tissue.
Example 23. The method according to example 1, wherein said acquiring is performed externally to the patient's body.
Example 24. The method according to example 1, wherein said acquiring is performed internally to the patient's body.
Example 25. The method according to example 24, wherein said acquiring is performed via a thermal camera mounted on an endoscope.
Example 26. The method according to example 25, wherein said acquiring is performed by inserting said thermal camera through at least one external body orifice.
Example 27. The method according to example 26, wherein said external body orifice comprises the vagina, anus, mouth, at least one nostril, at least one ear canal, and/or uretra.
Example 28. The method according to example 1, wherein said treated malignant tissue comprises a part or all of a breast and/or a part or all of a cervix.
Example 29. The method according to example 1, comprising: detecting at least one side-effect of said treatment based on said processed images.
Example 30. The method according to example 29, wherein said side effect comprises inflammation in said malignant tissue.
Example 31. A system for monitoring cancer treatment using thermography, comprising:
a thermal imaging camera suitable for acquiring thermal images of a tissue region in which malignant tissue is present;
a controller programmed to operate said camera one or more times throughout a treatment course according to one or more predefined protocols;
memory circuitry for storing one or more thermal images and/or processed images; and a processor configured to analyze the acquired thermal images for indicating the tissue response to treatment based on a condition of vasculature associated with said malignant tissue; wherein said processor compares said acquired thermal images to said stored thermal images and/or said stored processed thermal images.
Example 32. The system according to example 31, wherein said processor is programmed to apply one or more image processing algorithms designed to identify said vasculature condition or changes therein.
Example 33. The system according to examples 31 or 32, wherein said system is configured to provide a progress related indication for determining the efficacy of treatment.
Example 34. The system according to example 31, wherein said system is configured to be integrated in and/or added onto an irradiating modality.
Example 35. The system according to example 34, wherein said system is configured to automatically modify an irradiation scheme of said irradiating modality based on real time feedback obtained from said thermal images.
Example 36. The system according to example 31, wherein said camera is configured to acquire infrared images with a resolution of at least 320×256 pixels.
Example 37. The system according to example 31, wherein said thermal imaging camera is shaped and sized to be inserted through a body orifice.
Example 38. The system according to example 37, wherein said body orifice comprises the vagina, anus, mouth, at least one nostril, at least one ear canal and/or uretra.
Example 39. The system according to example 31, wherein said thermal imaging camera is shaped and sized to be inserted at least 5 mm into the body.
Example 40. The system according to example 31, wherein said tissue region comprises breast tissue region or cervical tissue region.
Example 41. A device for analyzing thermal images of a malignant tissue, comprising:
a memory for storing two or more thermal images, and/or processed thermal images of said malignant tissue; and
a control module configured to detect changes in a tumor and/or vasculature in said malignant tissue by comparing two or more of said stored thermal images and/or processed thermal images.
Example 42. The device of example 41, further comprising an interface circuitry, wherein said interface circuitry delivers an indication based on said detected changes.
Example 43. A method of characterizing a tumor, comprising:
acquiring one or more thermal images of a tissue region in which said tumor is found; processing said one or more thermal images to detect vasculature; and
analyzing the processed images to determine a condition of said tumor based on said vasculature.
Example 44. The method according to example 43, wherein said condition comprises one or more of a size, volume, spread, and stage of said tumor.
Example 45. The method according to example 43, wherein said tissue region comprises a part or all of a breast and/or a part or all of a cervix.
Example 46. The method according to example 43, wherein said analyzing comprising:
analyzing the processed images to locate areas of dense vasculature in said tissue region; and
detecting said tumor in said tissue region based on location of said dense vasculature.
Example 47. The method according to example 43, comprising determining if said tumor is a non-malignant tumor, a pre-malignant tumor or a malignant tumor based on said tumor condition.
Example 48. The method according to example 43, wherein said determine a condition of said tumor comprises determine the stage of said tumor.
Example 49. The method according to example 43, wherein said analyzing comprises quantifying an entropy level of said detected vasculature and wherein said condition of said tumor is based on said quantified entropy level.
Example 50. The method according to example 43, comprising: selecting a treatment protocol for treating said tumor based on the results of said characterizing.
Example 51. The method according to example 43, wherein said acquiring comprises acquiring one or more visible light images and said one or more thermal images of said selected tissue region.
Example 52. A method for detecting vasculature and/or tumor in a malignant tissue, comprising:
acquiring one or more thermal images of said malignant tissue;
applying a Frangi filter on said one or more thermal images to produce a filtered image of said malignant tissue;
detecting said vasculature and/or tumor in said filtered image.
Example 53. A method of diagnosing a patient predicted to develop radiation recall dermatitis, comprising:
determining temperature levels of a malignant tissue of said patient subjected to radiotherapy, wherein said malignant tissue exhibits an increase or no change in said temperature levels following said radiotherapy compared to temperature levels of said malignant tissue prior to radiotherapy.
Example 54. The method according to example 53, comprising:
selecting a treatment regime for treating said patient based on the results of said determining.
Example 55. The method according to example 53, comprising:
treating said patient based on the results of said determining.
Example 56. Chemotherapy for use in the treatment of cancer in a subject in need thereof, wherein said subject exhibits higher or stable temperature level of a malignant tissue subjected to radiotherapy, compared to the temperature level of said malignant tissue prior said radiotherapy.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

The following FIGS. 1-3 include images (raw and processed) collected during a clinical trial performed in accordance with some embodiments of the invention.

FIG. 1: Left: CT image taken before radiotherapy that was used to plan the treatment, according to some embodiments. The breast, tumor, and isodoses are marked, in accordance with some embodiments. Color wash, 90% isodose is shown in blue and 90-107% dose in yellow. Middle: Thermal image taken before radiotherapy, according to some embodiments. Temperature scale in the image is between 32 and 37.7° C. The red area indicates where the skin temperature exceeds 37.9° C. In some cases, for example as shown herein, a correlation exists between the warm area on the skin and the shape of the tumor on the CT. In some cases, for example as shown herein, the folds under the breasts are warmer and therefore their temperature exceeds 37.7° C. but this does not indicate a tumor. Right: A thermal image of patient no. 1 before radiotherapy on a color scale, according to some embodiments. The temperature scale in the image is 32-37.9° C.

FIG. 2: The top picture is a thermal image of patient no. 1 before beginning treatment, according to some embodiments. The left-hand panel shows a processing of the image of the tumor area, marked by the red box. The middle picture shows the same patient and type of image during radiotherapy. The bottom picture shows the same patient at the end of treatment.

FIG. 3: Thermography of patient no. 2. The top image shows before irradiation, the middle image after a total dose of 20 Gy, and bottom image after a total dose of 48 Gy. The temperature scale in the image is 32-39° C.

FIG. 4: is a schematic representation of the mechanisms potentially leading to a rise in breast temperature during radiotherapy [28], as evidenced from some of the cases in the clinical trial.

FIG. 5: is a flowchart of a general method for processing a thermal image, according to some embodiments of the invention. In some embodiments, the method is applied to highlight vasculature in the thermal image.

FIG. 6A: is a block diagram of a general system for monitoring a tissue response to cancer treatment, according to some embodiments of the invention;

FIG. 6B: is a detailed diagram of components and operation of a system for monitoring cancer treatment, according to some embodiments of the invention;

FIG. 6C: is a flow chart of a process for tumor detection and staging based on thermography results, according to some embodiments of the invention;

FIG. 6D: is a flow chart of a process for determining treatment efficacy based on thermography results, according to some embodiments of the invention;

FIG. 6E: is a flow chart of a process for characterizing a tumor and/or a patient following treatment based on thermography results, according to some embodiments of the invention;

FIG. 7A: is a table summarizing the characteristics, treatment and outcome of patients 1-6 that participated in a clinical trial for analyzing thermal images to monitor radiotherapy, according to some embodiments of the invention;

FIG. 7B: is a graph of the changes in delta temperature of patients 1 to 6 during a radiotherapy treatment, according to some embodiments of the invention;

FIG. 7C: is a graph of the changes in maximal temperature of patients 7 to 14 during a radiotherapy treatment, according to some embodiments of the invention;

FIG. 8A: is a CT scan of a patient with viable tumor contoured in the right breast (blue line), according to some embodiments of the invention;

FIG. 8B: is a thermal image of a tumor area shown in FIG. 8A, according to some embodiments of the invention;

FIG. 8C: is a table summarizing the reduction in tumor signal following radiotherapy in patients 1 to 6, according to some embodiments of the invention;

FIG. 8D: is a processed thermal image of a tumor before, during and after radiotherapy, according to some embodiments of the invention;

FIG. 8E: is a flow chart of a process for detecting changes in vasculature, according to some embodiments of the invention;

FIG. 9A: is a table summarizing the characteristics and treatment details of patients 1 to 6 that underwent brachytherapy, according to some embodiments of the invention;

FIG. 9B: is a PET-CT scan of a cervix tumor, according to some embodiments of the invention;

FIG. 9C: is a thermal image of a cervix tumor, according to some embodiments of the invention;

FIG. 9D: is a graph depicting the change in delta temperature between the maximal and minimal temperatures of the cervix during brachytherapy in patients 1 to 6 (patient 4 is excluded), according to some embodiments of the invention;

FIG. 10A: is a flow chart describing the process of thermal images analysis using an algorithm, according to some embodiments of the invention;

FIG. 10B: is a detailed flow chart describing the different steps of a process for analysis of thermal images using the algorithm, according to some embodiments of the invention;

FIG. 10C: is an image describing the preprocessing step of the algorithm, according to some embodiments of the invention;

FIG. 11: is an image describing the reduction in tumor and vasculature signals during radiotherapy, according to some embodiments of the invention;

FIGS. 12A and 12B: are images of PET-CT scans taken before (12A) and after treatment (12B), according to some embodiments of the invention;

FIGS. 12C and 12D: are images describing the tumor density and entropy before (12C) and after treatment (12D), according to some embodiments of the invention; and

FIG. 13: is a table summarizing the change in entropy after treatment for patients 1-6, according to some embodiments of the invention.

FIGS. 14-19: are tables (labeled Tables 1-6, respectively) which present treatment details and temperature data collected during a clinical trial, performed in accordance with some embodiments of the invention.

### DESCRIPTION OF SPECIFIC EMBODIMENTS OF THE INVENTION

The present invention, in some embodiments thereof, relates to monitoring cancer treatment and, more particularly, but not exclusively, to use of thermography as a tool for assessing cancer treatment, for example treatment efficacy and/or progress. Some embodiments of the invention relate to use of thermography as a tool for monitoring and/or characterizing tumor grading and/or staging.

An aspect of some embodiments relates to thermally imaging tissue to detect vasculature associated with malignant tissue and/or changes in vasculature. In some embodiments, the vascular condition and/or changes therein provide an indication of the tissue response to treatment. In some embodiments, the term vasculature defines the arteries, capillaries and veins that supply blood to and from the malignant tissue.

In some embodiments, one or more thermal images are acquired before, during and/or after a treatment course in which a patient is treated by irradiation and/or chemotherapy and/or hormonal treatment. Optionally, images are acquired before, during and/or after irradiation sessions performed during the treatment course. For example, thermal images may be acquired before, during and/or after 1, 2, 5, 7, 10 or intermediate, higher or lower number of irradiation sessions performed during a treatment course. In some embodiments, the full treatment course ranges between, for example, 1 week to 3 weeks, 2 weeks to 10 weeks, 5 weeks to 20 weeks or intermediate, longer or shorter time periods, and thermal images are acquired once every week, twice every week, 5 times a week, or intermediate, higher or lower number of times.

In some embodiments, images are acquired at a plurality of predetermined timings throughout the treatment course. Optionally, the timings are selected in accordance with one or more parameters of a treatment regimen, for example in accordance with radiation and/or chemotherapy dosing.

In some embodiments, the acquired thermal images are processed to identify a physiological state and/or process in the tissue, such as a current vasculature condition and/or changes in vasculature. In some embodiments, the images are processed to accentuate blood vessels and/or capillaries associated with a tumor, such as vessels that supply blood to the tumor, vessels that form a part of the tumor.

In some embodiments, a condition of the tumor is deduced from the processed image (e.g. tumor size, volume, spread and/or location). In some embodiments, a tumor's stage is deduced from the processed image. In some embodiments, the tumor stage is deduced by combining vasculature related data and tumor related data collected from the processed thermal image. In some embodiments, the tumor stage is deduced by comparing the collected data to a database and/or reference table. Optionally, the tumor stage is deduced by comparing to pathology results and/or results obtained using other methods and/or modalities, e.g. CT.

In some embodiments, the tumor's growth rate (e.g. tumor doubling time) is deduced from the processed image, for example by comparing two or more images obtained at different times.

In some embodiments, a condition of the vasculature associated with the tumor is deduced from the processed image. In some embodiments, an inflammatory condition of the tissue is deduced from the processed image.

In some embodiments, the results of processing the image are calibrated, for example in reference to one or more additional images acquired from the patient and/or in reference to a database.

In some embodiments, changes in vasculature are identified by comparing a thermal image to one or more previously acquired images of the same tissue region. In some embodiments, changes in vasculature such as a reduced number of vessels and/or capillaries, reshaped vessels, a reduced vessel density, a change in vessel diameter and/or other changes are indicative of a reduction in a tumor's size and/or volume and/or malignancy. In some embodiments, changes in vasculature that are indicative of radiation induced tumor endothelial cell death are assessed. As apoptosis of tumor endothelial cells may lead to apoptosis of tumor parenchymal cells, assessment of radiation induced endothelial cell death by analyzing changes in vasculature may contribute to determining the radiotherapy efficacy.

In some embodiments, the treatment does not include a vasculature-targeted treatment. In some embodiments, the treatment is selected to target cells (e.g. malignant tissue cells) and the effect of such treatment is deduced, according to some embodiments, from a vascular condition of the treated tissue.

In some embodiments, thermal images acquired over the treatment course are compared to each other to determine changes in temperature distribution. In some cases, temperature changes are associated with treatment, for example a temperature decrease may be indicative of a reduction in the tumor's malignancy following irradiation; a temperature increase may be indicative of an inflammatory response in the tissue, for example following irradiation and/or resection of the tumor; and/or other changes associated with treatment.

In some embodiments, a temperature drop in the target tissue (in which the tumor is present) is indicative of a reduction in the tumor's heat production capabilities. In some cases, such as before treatment, the tumor tissue exhibits a higher temperature than surrounding tissue. Optionally, a decrease in the temperature difference between the tumor tissue and the surrounding tissue is indicative of a positive response of the tumor to treatment.

In some cases, a rise in temperature due to inflammation is associated with vessel dilation.

In some embodiments, a temperature distribution of a first tissue region (e.g. target tissue region, in which malignant tissue is present) is normalized with respect to a second tissue region (e.g. non-targeted region). For example, when treating breast cancer, a temperature distribution of the target breast is normalized with respect to the temperature distribution of the non-targeted breast. A potential advantage of normalizing the temperature distribution may include eliminating environmental factors (e.g. room temperature). In some cases, a temperature drop in the normalized temperature of the treated tissue is indicative of an effective treatment.

In some embodiments, an average, maximal and/or minimal temperature of the targeted tissue (e.g. breast) or portions thereof (e.g. nipple) is calculated from the thermal image. In some embodiments, a similar parameter (e.g. average, maximal and/or minimal temperature) of non-target tissue or portions thereof used as reference is calculated (e.g. the non targeted breast). A potential advantage of referring to the nipple temperature may include that the nipple tissue may reflect environmental temperature effects more than the surrounding skin tissue, allowing to take those effects into consideration.

In some embodiments, a threshold is applied, for example to distinguish between temperature changes associated with treatment effects and other temperature changes (e.g. random changes or changes associated with non-related physiological conditions). In some embodiments, the applied threshold comprises the temperature of the untreated breast, for example the breast that was not subjected for radiation therapy, or other types of therapy.

In some embodiments, spatial variations in the temperature distribution are assessed. Optionally, a decrease in the size of a skin region in which high temperatures were detected may be indicative of a reduction in the tumor size. In some cases, treatment is effective to reduce tumor metabolic heat production, which in turn affects a size of the tumor as reflected by the tissue surface temperature distribution.

In some embodiments, the concentration or density of blood vessels is determined based on the acquired thermal images. Optionally, by analyzing the blood vessels concentration or density, pre-malignant, early stage malignant, and/or malignant tumors are detected. In some embodiments, the detected tumors are breast cancer tumors and/or cervix cancer tumors.

In some embodiments, the acquired thermal images are used to detect blood vessels having a diameter of at least 15 μm, for example 15, 50, 100, 500 μm or any intermediate or larger values. In some embodiments, the acquired thermal images are used to detect individual small blood vessels having a diameter of at least 15 μm, for example 15, 50, 100, 500 μm or any intermediate or larger values. In some embodiments, the number of blood vessels and/or the density of blood vessels and/or the average diameter of blood vessels in a selected region are determined based on the acquired thermal images. In some embodiments, the change in blood vessel number and/or the change in blood vessel density and/or the change in the average blood vessel diameter are determined based on the acquired thermal images.

A potential advantage of monitoring treatment such as radiotherapy using thermography may include the ability to identify, optionally in real time, ongoing processes and/or anatomical changes in the tissue, such as changes in tumor vasculature. Another potential advantage of monitoring radiotherapy using thermography may include using a simple, available, non-contact, non-irradiating tool.

An aspect of some embodiments relates to a system configured for monitoring cancer treatment using thermography. In some embodiments, the system is configured for detecting vasculature associated with malignant tissue and/or changes therein by analyzing a temperature distribution of the tissue. In some embodiments, the system is configured to provide a progress-related indication, for example an indication related to decline in the heat production of the tumor and/or tissue related to the tissue, for determining the effectiveness of treatment (e.g. radiotherapy and/or chemotherapy).

An example for early detection of response to therapy and possible early change in treatment is early detection locally advanced breast cancer, treated with neoadjuvant chemotherapy (prior to surgery, to reduce tumor size). If the chemotherapy is not effective enough, we will not continue the whole 4 cycles regimen, and it will be changed to another chemotherapy agents, that will be more effective.

In some embodiments, the system delivers an indication related to changes in the tumor, for example changes in tumor size, volume, shape, and or stage.

In some embodiments, the system delivers a different indication related to changes in vasculature outside the tumor or inside the tumor, for example changes in vascular density, distribution, and/or blood vessel diameter average.

In some embodiments, the system delivers a combined indication for changes in the tumor and changes in the vasculature.

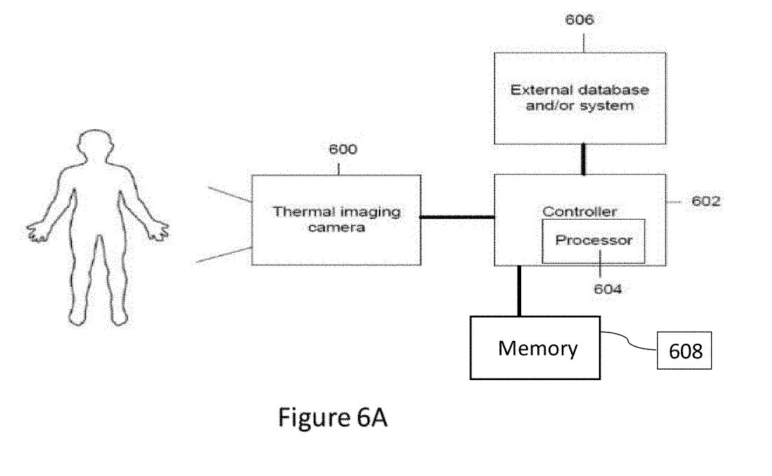

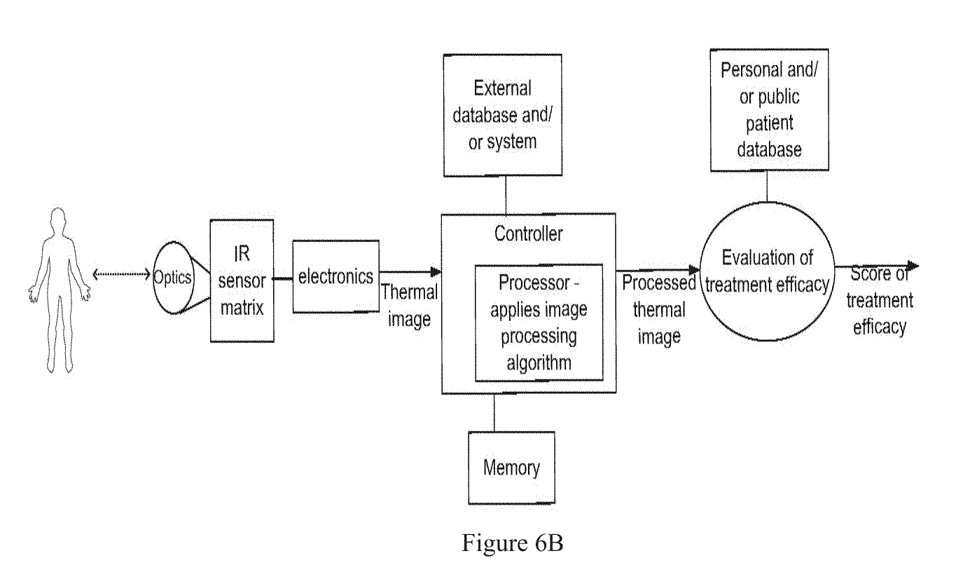

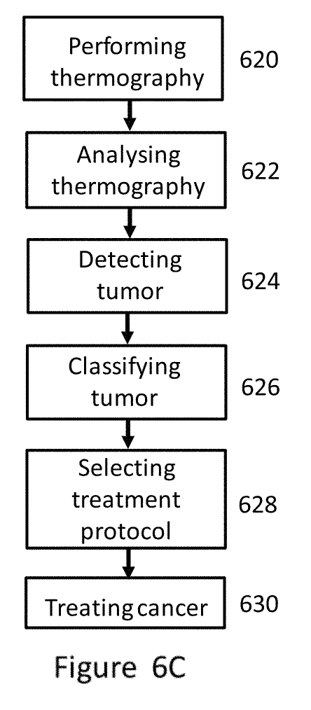

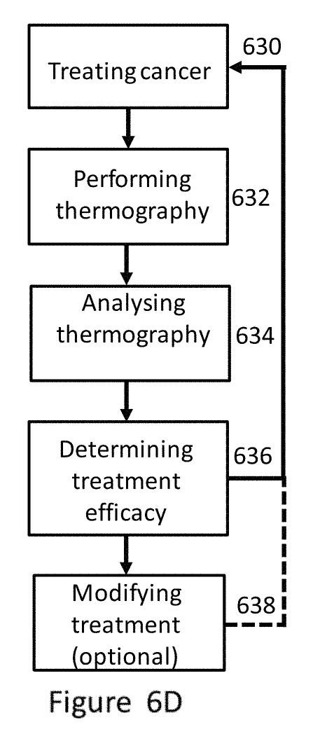

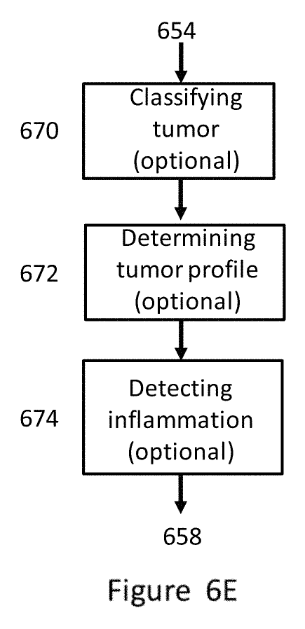

In some embodiments, for example as schematically illustrated in FIG. 6A, the system comprises a thermal imaging camera (600) suitable for acquiring thermal images of the tissue undergoing treatment. In some embodiments, the camera is suitable to detect infrared radiation emitted from the patient's skin surface, at wavelengths of, for example, between 0.8 μm and 1 μm. Exemplary camera parameters may include an infrared resolution of, for example, 100-1000×100-1000 pixels, an image frequency of between 10-100 Hz and thermal sensitivity of, for example, less than 0.05° C., less than 0.1° C., less than 0.5° C. or intermediate, higher or lower values.

In some embodiments, the system comprises a controller (602) programmed to acquire the images via the camera according to one or more protocols. In some embodiments, the controller is programmed to acquire images at a plurality of pre-determined timings. Optionally, the predetermined timings are selected in accordance with the treatment regimen, for example according to the dosing and/or according to supplementary medication prescribed to the patient and/or according to expected changes in the tissue and/or total patient condition.

In some embodiments, the system comprises a processor (604) configured for processing the acquired images. Optionally, the processor forms a part of the controller. In some embodiments, the processor is configured to apply one or more image processing algorithms are applied to the acquired images.

In some embodiments, the system comprises a memory (608), connected to the controller (602) or processor (604). In some embodiments, memory (608) stores at least one algorithm of the image processing algorithms or part of an algorithm. Additionally, memory (608) stores at least one thermal image, and/or at least one processed thermal image and/or results of at least one image processing procedure. In some embodiments, memory (608) stores at least one treatment plan, treatment plan parameters and/or values of treatment plan parameters.

In some embodiments, the applied algorithm is designed for highlighting vessels associated with a tumor. In some embodiments, the algorithm is designed to detect narrow vessels, bending vessels, branching vessels, a high vessel density, and/or other vessel irregularities which may be associated with vasculature leading to, into and/or from the tumor. In some embodiments, the applied algorithm detects narrow vessels, having a diameter which is less than 50% of the diameter of the largest vessel in the analyzed region, for example 50%, 40%, 30%, 20% or any intermediate or lower value. In some embodiments, the applied algorithm detects bifurcation or branching of blood vessels into two or more branches, optionally by detecting the branching points.

In some embodiments, the applied algorithm is used for detecting tumors having a size of at least 0.5 cm, for example 0.5, 1, 1.5 cm or any intermediate or larger size. In some embodiments, the applied algorithm is used for detecting tumors having at least one dimension, for example height, width and/or length with a length of least 0.5 cm for example 0.5, 1, 1.5 cm or any intermediate or larger size.

In some embodiments, the applied algorithm is designed for detecting a location and/or size and/or malignancy level of a tumor. Optionally, the tumor appears as a gleaming white spot in the processed images. In some cases, a reduction in the brightness of the spot is indicative of a reduction in the tumor malignancy in response to treatment.

In some embodiments, the applied algorithm is designed for masking thermal effects resulting from inflammation of the tissue, for example so that inflammation does not interfere with assessment of vasculature. Additionally or alternatively, the applied algorithm is designed for detecting and optionally monitoring inflammation. A potential advantage of monitoring inflammation may include improving a patient's prognosis.

In some embodiments, the applied algorithm is designed for distinguishing between tissue regions that exhibit a high temperature due to the presence of a tumor, tissue regions that exhibit a high temperature due to inflammation, and/or normal tissue regions that exhibit a high temperature due to their location, such as a tissue fold (e.g. a tissue fold under the breast).

In some embodiments, the applied algorithm takes into consideration an anatomy of the imaged tissue and thermal effects which may result from that anatomy. For example when imaging breast tissue, a tissue fold under the breast may be naturally warmer than surrounding tissue, and the algorithm will identify that fold in the image and analyze the temperature distribution accordingly.

In some embodiments, the system receives as input a certain anatomy (e.g. an anatomy including a tissue fold) and/or expected heat distribution that is taken into consideration when processing the image. Additionally or alternatively, borders between different organs and/or tissue types are recognized during processing of the image and are taken into consideration. In some embodiments, the applied algorithm takes into consideration a geometry and/or a specific location of the tumor relative to surrounding tissue or organs. For example, if a tumor protrudes outwardly relative to the skin surface, it may be cooler as compared to, for example, a tumor underlying the surface, and the analysis will be performed under that assumption.

In some embodiments, the applied algorithm takes into consideration tissue regions (and/or outlines of those regions) that are naturally shadowed when the image is taken, such as a chest area covered by the breast.

In some embodiments, the system is configured for external imaging, such as for imaging the breast, head and/or neck regions, skin, anal region, cervix and/or other externally approachable areas. Alternatively, the system is configured to internal imaging, for example using a thermal camera mounted on an endoscope. Such configuration may be advantageous, for example, when treating tumors located at a depth from the skin surface. Optionally, the system configured for internal imaging is used when internal irradiation is applied, such as by a radioactive capsule.

In some embodiments, the system is configured to provide a progress-related indication to the physician and/or other clinical personnel. The physician may decide to modify the treatment regimen in view of the provided indication (e.g. change the doses administered and/or timing thereof; prescribe medication; and/or other).

In some embodiments, the treatment efficacy is quantified, for example according to an index. Optionally, the system is configured provide a measure of efficacy of the applied treatment. For example, the system may be configured to indicate that a certain irradiation session performed achieved a certain percentage of its expected therapeutic effect. In some embodiments, the efficacy is quantified with respect to previous measurements performed. In some embodiments, the efficacy is quantified by comparing to measurements obtained using other modalities and/or methods.

In some embodiments, the system is configured to be integrated in and/or in communication with an irradiating modality (e.g. a linear accelerator), mammography device and/or other devices used for treating and/or for monitoring treatment. In some embodiments, the system is configured to automatically modify an irradiation scheme of the irradiating modality based on feedback obtained from the acquired thermal images. Optionally, modification of the irradiation scheme is performed in real time, for example during an irradiation session.

In some embodiments, the controller (optionally including the processor) is configured for remote operation of the camera. Alternatively, the controller is configured locally.

In some embodiments, the controller (602) is in communication with an external database and/or system (606). The database may include, for example, reference thermal images, previous results of the patient and/or other patients, and/or other data. In some embodiments, the external system comprises a hospital system.

In some embodiments, for example as shown in the diagram of an exemplary system of FIG. 6B, the system is configured to receive and/or acquire a thermal image as input, and to provide an evaluation of treatment efficacy (for example a score of efficacy) as output. In some embodiments, a thermal image obtained using infrared imaging means is processed by applying one or more image processing algorithms for example as described herein below.

In some embodiments, the processed image is analyzed to evaluate the efficacy of treatment according to one or more indications of the tissue response to the treatment, deduced from the processed image. Optionally, evaluation comprises comparing results to a personal database, including, for example, previous results of the patient, such as results of previous treatment sessions (e.g. irradiation and/or chemotherapy sessions). Additionally or alternatively, the results are compared to a public database, including, for example, results collected from other patients and/or results associated with a certain pathology or condition. In some embodiments, the results are compared to data stored in memory, for example memory 608.

An aspect of some embodiments relates to a personal follow-up device configured for thermally imaging the tissue of a patient that underwent cancer treatment, including, for example, radiotherapy and/or chemotherapy. In some embodiments, the device is configured to provide an indication related to recurrence of the disease, such as an indication related to existence of malignant tissue and/or other findings detectable by analyzing the skin temperature distribution. In some embodiments, the device comprises an IR camera and a control module. Optionally, the camera is configured to be attached to a smartphone. In some embodiments, the device communicates with a designated application suitable for presenting the acquired images and/or analysis thereof to the patient. In some embodiments, the device is configured for sending an alert to the physician to notify of suspicious findings and/or processes in the tissue, such as growth of vasculature.

An aspect of some embodiments relates to detecting cancer and/or monitoring a cancer treatment by thermal imaging of malignant tissue located inside the body from outside the body. Optionally, the cancer is detected and/or the cancer treatment is monitors from within the body. In some embodiments, cancer is treated and/or a cancer treatment is monitored by performing thermal imaging through an orifice of the body. In some embodiments, at least part of a thermal imager is introduced through an orifice of the body, for example through the vagina, anus, mouth, ear, at least one nostril, at least one ear canal and/or through the urethra. Optionally, the thermal imager is part of an endoscope. In some embodiments, the thermal camera, for example an IR camera is located at a distal end facing the tissue of an endoscope. In some embodiments, thermal imaging from within the body allows to, for example to thermally visualize tumors positioned inside the body, for example tumors of cervix cancer, colon cancer and/or laryngeal cancer.

In some embodiments, the thermal camera is positioned outside a body orifice. In some embodiments, the thermal camera acquires thermal images of a tissue located within the body, through the body orifice. Optionally, the tissue is manipulated to position at least part of the tissue, for example a tumorigenic part in the detection field of the external thermal camera. Alternatively, the camera is coupled to a thermal imaging bundle that enables the collection of thermal images from within body cavities, when inserted through the natural body orifices.

An aspect of some embodiments relates to determining the efficacy of a cancer treatment using radiotherapy. In some embodiments, treatment efficacy is determined based on thermal images of the tumor and/or vasculature associated with the tumor. Optionally, the treatment efficacy is determined based on thermal images of the tumor and/or vasculature associated with the tumor following the treatment. In some embodiments, if the treatment efficacy is not a desired treatment efficacy then the treatment protocol or a value of at least one treatment parameter is modified.

According to some embodiments, the efficacy of the cancer treatment is determined based on the temperature of the tumor and/or vasculature associated with the tumor following treatment. In some embodiments, the treatment efficacy is determined by monitoring the change in temperature of the tumor and/or vasculature associated with the tumor during the treatment, optionally compared to the temperature of the tissue before the treatment.

In some embodiments, a cancer treatment is considered to be efficacious when the temperature of the tumor and/or the reduction in vasculature associated with the tumor reduces in at least 2%, for example 2, 3, 4, 5% or any intermediate or larger value, after an accumulative radiation dose, for example 2, 10, 30 Gy or any intermediate or larger radiation dose. In some embodiments, the cancer treatment comprises radiotherapy, brachytherapy, chemotherapy or an immunotherapy treatment.

A possible advantage of using thermography for determining the efficacy of a treatment is that it allows to obtain information about the efficacy of the treatment, for example radiotherapy at a very early stage, before changes are evident in the size of the tumor or when changes are evident but are not associated with the treatment efficacy. Additionally, thermography enables to visualize physiological processes, for example the density and/or shape of vasculature near the tumor, and/or the tumor's heat production and not like other imaging techniques such as CT and MRI that only show the size of the tumor and not the physiological processes occurring before tumor size changes. Moreover, CT and MRI are more expensive and less readily available than thermography. Assessment of the efficacy of radiotherapy during treatment may promote changes in the treatment regimen, the dose, and the radiation field during therapy; and contribute to the determination of individualized treatment schedules for optimal treatment effectiveness.

An aspect of some embodiments relates to characterizing a tumor using thermography. In some embodiments, thermography is used for early detection and/or characterization of a tumor, optionally in combination with optical imaging or other imaging techniques. In some embodiments, a tumor is characterized prior to a treatment, for example to select a treatment protocol. Alternatively or additionally, the tumor is characterized using thermography during or following a treatment.

According to some embodiments, thermography is used to determine tumor staging and/or changes in tumor staging before or during treatment, optionally according to the TMN staging system. In some embodiments, thermography is used to stage a tumor as a pre-malignant or as an early malignant tumor, for example by detecting blood vasculature associated with the tissue. Optionally, early detection of a cancer, for example breast or cervix cancer, at an early stage using thermography allows better prognosis.

According to some exemplary embodiments, early detection of tumors using thermography allows to detect tumors at an early stage. In some embodiments, detecting an early stage tumor optionally allows better chances for tumor treatment, and optionally using less aggressive therapies.

An aspect of some embodiments relates to detecting at least one side-effect of the cancer treatment, for example an inflammation process in the tumor area using thermography. In some embodiments, the inflammation is detected by monitoring temperature of the tumor and/or temperature in the vasculature associated with the tumor during a treatment. In some embodiments, the inflammation is detected by monitoring temperature changes of the tumor and/or temperature changes in the vasculature associated with the tumor during a treatment. Optionally, the changes in vasculature temperature are caused by changes in the blood vessels. In some embodiments, the vasculature associated with the tumor is located outside the tumor and/or inside the tumor. Optionally, the temperature of the tumor and/or the vasculature after the treatment is compared to the temperature before the treatment. In some embodiments, during the inflammation process, induced by the treatment, the blood vessels are damaged, the cells that line the lumen are less adhered to each other. In some embodiments, the result is leakage, no appropriate blood supply and less oxygen delivered to the tumor. Local edema and skin damage occur, over prior irradiated area.

According to some embodiments, inflammation is detected when the temperature of the tumor and/or the vasculature increases or remains stable following treatment, compared to the temperature before the treatment. In some embodiments, the risk of developing radiation recall dermatitis following radiotherapy is predicted using thermography. In some embodiments, the risk of developing radiation recall dermatitis is increased when the temperature of the tumor and/or the vasculature increases or remains stable following radiotherapy.

According to some embodiments, if inflammation is detected then the cancer treatment is modified or replaced. Optionally, if the development of radiation recall dermatitis is predicted, then the radiotherapy treatment is modified or replaced by chemotherapy or immunotherapy or other anticancer agents.

According to some embodiments, Radiation recall phenomena is a rare, unpredictable, acute inflammatory reaction over the skin, confined to previously irradiated areas that can be triggered when certain anticancer agents, (i.e. Doxorubicin, 5-fluorouracil, cisplatin, cyclophosphamide, docetaxel, epirubicin, gemcitabine, trastuzumab) are administered after radiotherapy. For example, Doxorubicin and cisplatin are very common chemotherapeutic agent, often used in cancer patients, and the risk of developing radiation recall is higher when they are used. Other agents are for example: 5-fluorouracil, cyclophosphamide, docetaxel, epirubicin, gemcitabine, trastuzumab. If radiation recall phenomena is anticipated, the oncologist may choose a different anticancer agent.

A possible advantage of using thermography is the ability to obtain information about the inflammation process at a very early radiation dose, before changes are evident with any other methods, enabling early prediction of late consequences and may lead to dose reduction as needed. Optionally controlling this inflammation process allows to increase the efficacy of the treatment.

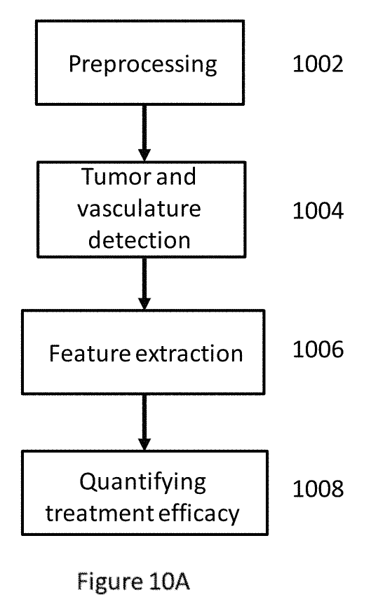

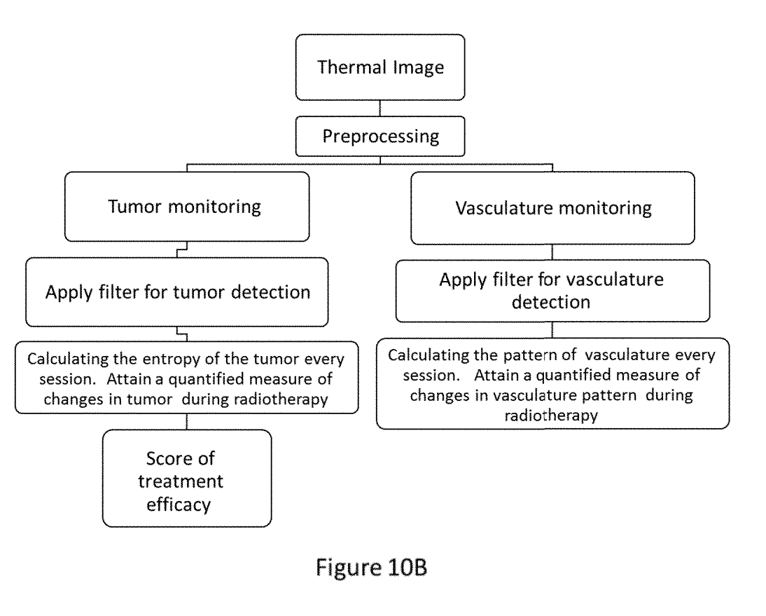

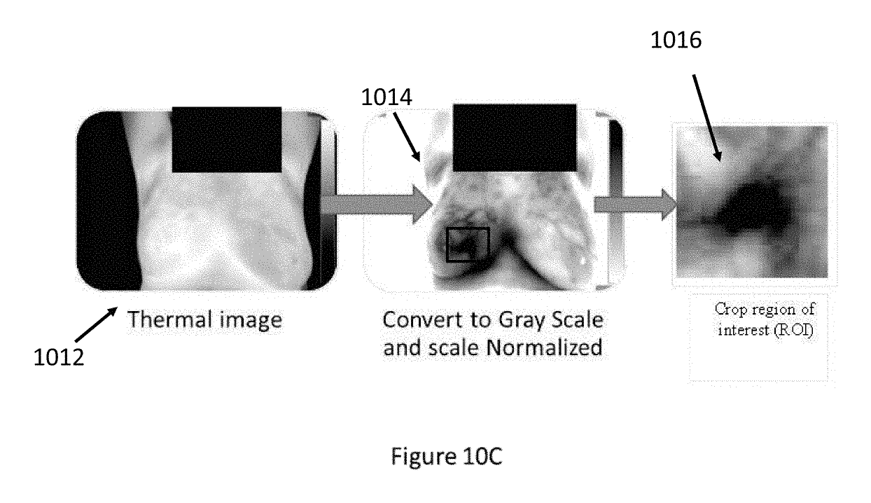

An aspect of some embodiments relates to detection tumor and/or vasculature by application of Frangi filter (Multiscale vessel enhancement filtering Alejandro F. Frangi, Wiro J. Niessen, Koen L. Vincken, Max A. Viergever) on thermal images of a malignant tissue. In some embodiments, the Frangi filter is applied after processing of the thermal images, for example after filtering and/or after a region of interest (ROI) is selected. In some embodiments, the Frangi filter is applied, for example as described in FIGS. 10A and 10B.

According to some exemplary embodiments, the Frangi filter is applied by a device configured to process one or more thermal images. In some embodiments, the device comprises a memory circuitry which stores at least one algorithm and/or at least one filter. Additionally, the memory stores at least one thermal image and/or at least one processed thermal image.

According to some exemplary embodiments, the device used for processing one or more thermal images comprises a control module. In some embodiments, the control module detects a tumor and/or vasculature in the malignant tissue. In some embodiments, the tumor and/or vasculature is detected after the application of the Frangi filter. In some embodiments, the device comprises an interface circuitry functionally connected to the control module. In some embodiments, the control module signals the interface circuitry to generate an indication, for example a human detectable indication if a tumor is detected in the tumorigenic tissue, optionally after the application of the Frangi filter.

While some embodiments are described with respect to monitoring of breast cancer treatment, it is noted that methods and/or devices for example as described herein may be used for monitoring treatment of tumors (or other malignant tissue) of body systems and/or organs other than breast, such as head and neck, cervix, anal region and/or other.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

### Exemplary Monitoring Breast Cancer Treatment by Thermography

### Summary

Breast cancer is the most frequently diagnosed cancer among women in the Western world. Thermography, a non-ionizing, non-invasive, and low-cost method based on the detection of mid-IR radiation inertly emitted from the surface of a measured object, is an imaging modality that was traditionally used to detect breast cancer tumors but has not been examined as a treatment monitoring tool, in accordance with some embodiments of the invention. The clinical study described herein is an example of using thermal imaging as a tool for cancer treatment monitoring, according to some embodiments of the invention. In the clinical study, patients were monitored by imaging with a thermal camera prior to radiotherapy sessions over several weeks throughout the treatment period. In some embodiments, one or more thermal images are acquired and analyzed to detect a response of the tissue to treatment, such as radiotherapy and/or chemotherapy.

Radiation-induced endothelial cell death may affect the efficacy of treatment, in accordance with some embodiments. Some embodiments of the invention, as described for example in this study, relate to assessing vasculature changes using thermal imaging. In some embodiments, assessing the efficacy of radiotherapy during treatment makes it possible to change the treatment regimen, dose, and/or radiation field during treatment as well as to individualize treatment schedules to optimize treatment effectiveness.

### Introduction

Breast cancer, the most frequently diagnosed cancer among women in the Western world [1], can be imaged by any of several modalities, such as computed tomography (CT), MRI, and PET. All of these modalities measure a tumor's size and location [1-3], but their use is limited by their availability and cost.

Thermography, a non-ionizing, non-invasive, and low-cost method based on the detection of mid-IR radiation inertly emitted from the surface of a measured object [4], is an imaging modality that was traditionally used to detect breast cancer tumors [5-10], is explored herein as a treatment monitoring tool, according to some embodiments. Any object with a temperature above absolute zero emits radiation from its surface. Thermography allows the temperature distribution of an object to be recorded using the infrared radiation emitted by the surface of that object at wavelengths between 8μm and 10 μm [11], in accordance with some embodiments.

Emissivity is a measure of the efficiency at which a surface emits thermal energy. It is defined as the fraction of energy being emitted relative to the energy emitted by a thermally black surface (a black body). A black body is a material that is a perfect emitter of heat energy, with an emissivity value of 1. Because human skin has a high emissivity, 0.98, measurements of infrared radiation emitted by human skin can be converted directly into accurate temperature values. The high sensitivity of thermography to surface changes may be advantageous in cancer treatment monitoring. In some embodiments, monitoring using thermography is based on the assumption that malignant tumors are characterized by abnormal metabolic and perfusion rates [11, 12], and are therefore expected to show an abnormal temperature distribution compared with the surrounding healthy tissue [13, 14]. In some embodiments, a known correlation between metabolic heat production and tumor growth [15, 16] is taken into consideration; the higher the tumor malignancy, the more heat it produces [16, 17]. Therefore, at least in some cases, a change in skin temperature during treatment may provide a measure of the tumor's response to treatment.

While thermography has been extensively researched as a breast cancer detection tool [3-8], its use to monitor treatment has never been evaluated. In the exemplary study described herein, the feasibility of using thermography as a breast cancer treatment monitoring tool is explored. To monitor treatment efficacy, thermographic measurements were compared to clinical assessments during the course of radiotherapy, to evaluate the possibility of using thermography as a monitoring tool, according to some embodiments.

### Methods

Five radiotherapy patients participated in this clinical trial. A physician examined each patient and compiled the medical history and current complaints. The option of thermographic monitoring was explained to the patient and she was asked to participate in the research. If she agreed, she signed a consent form. Subjects were required to provide informed consent prior to participation.

Patients were monitored using a thermal camera throughout the radiotherapy period, according to some embodiments. The purpose of this exemplary study was to investigate the possibility of using thermal imaging as a tool for real-time feedback for cancer treatment and monitoring, according to some embodiments. Images of the patients were regularly taken before radiotherapy treatment sessions over a period of several weeks, according to some embodiments. The infrared camera used was a FLIR A35 (Boston, Mass.), which has an infrared (IR) resolution of 320×256 pixels with an image frequency of 60 Hz and object temperature range of −40° C. to 160° C. (It is noted that cameras or other thermal imagers suitable for acquiring images of tissue may be used. The above described specifications are not limiting). To maintain fixed environmental conditions, the room temperature was set to 24-26° C. and the room humidity to 50-57%. In addition, fluorescent lamps were turned off during image acquisition.

The thermal images taken during radiotherapy were analyzed using the FLIR Tool software (ResearchIR), which calculated the maximal and average temperatures of the breast tissue, according to some embodiments. For patient no. 1, who had an active tumor, the images of the breast obtained during radiotherapy treatment were processed by an algorithm that highlights blood vessels with malignant properties, according to some embodiments. In some cases, a prolific network of blood vessels develops around tumors. In some cases, tumor blood vessels are irregular in diameter with rather narrow tubes; in some cases, the capillaries are sharply bent, winding, and/or branched with multiple dead ends [18, 19]. In some cases, normal tissues have a well-organized network of homogeneous capillaries [20-22].

In the exemplary clinical study described herein, MATLAB based functions were applied for processing the images. It is noted that algorithms for example as described herein may be carried out by other suitable programs and/or tools.

### Results

### Patient Treatment and Imaging:

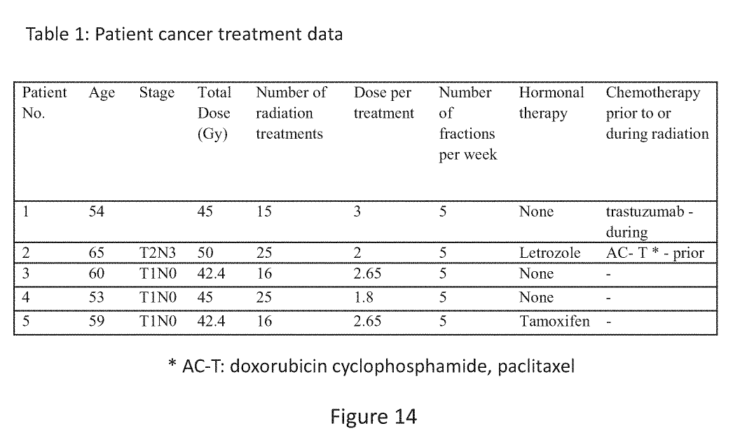

In this exemplary study, the breast skin temperature of five women undergoing radiotherapy was monitored, in accordance with some embodiments. Four patients received radiotherapy after undergoing tumor resection. In these patients, the purpose of the radiotherapy was to prevent disease recurrence. Patient no. 1 was 54 years old and has stage 4 breast cancer. She received 45 Gy of radiotherapy, divided into 15 sessions of 3 Gy per session. The treatments were administered 5 days a week, Sunday through Thursday, for 3 weeks in total. During radiotherapy, patient no. 1 also received trastuzumab. Her breast volume was 953.3 cc, the tumor volume was 24 cc, and the tumor depth began at the skin surface and reached a depth of 6 cm. Pertinent patient clinical information is presented in FIG. 14 (Table 1).

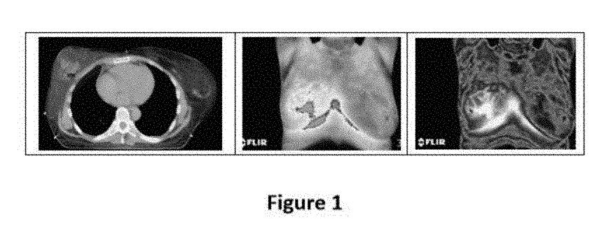

FIG. 1 shows images obtained from patient no. 1. The picture on the left is of a CT image taken prior to the radiotherapy; the middle shows a thermal image taken prior to radiotherapy, in accordance with some embodiments. The red area indicates skin temperatures exceeding 37.7° C. A correlation is assumed between the hot area on the skin and the size of the tumor in the CT. In some patients, folds under the breasts are warmer, and therefore the temperature in those areas may exceed 37.7° C., but this temperature elevation does not indicate a tumor. The picture on the right shows a thermal image of patient no. 1 prior to treatment on a color scale, according to some embodiments.

Patients 2-5 underwent tumor resection. Their cancer treatment data (radiotherapy, chemotherapy, and hormonal therapy) is presented in FIG. 14 (Table 1). Before she developed breast cancer, patient no. 4 had undergone breast augmentation surgery with silicone implants in both her breasts. The implant remained in her breast but the tumor was resected. In order to protect the implants, a lower dose of radiation per fraction was administered.

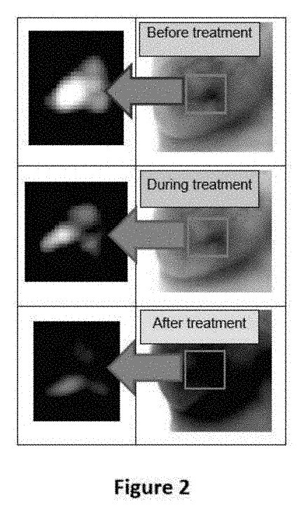

FIG. 2 shows the thermal imaging of patient no. 1 before, during, and after treatment, in accordance with some embodiments. The left-hand panel shows the tumor area, highlighted by the red box in the main image, after image processing, in accordance with some embodiments. On the top thermal image, taken prior to beginning treatment, after image processing it is possible to see the concentration of blood vessels with malignant properties, in accordance with some embodiments. In the middle thermal image, taken during radiotherapy in accordance with some embodiments, the vasculature is visibly reduced. In the bottom thermal image, taken at the end of treatment in accordance with some embodiments, a sharp decrease in the concentration of blood vessels with malignant properties is evident.

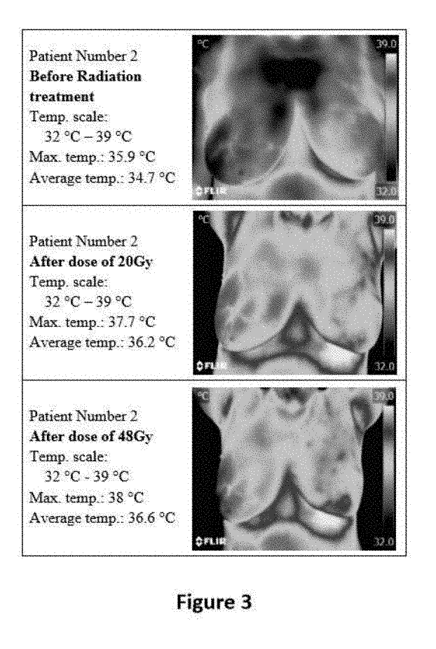

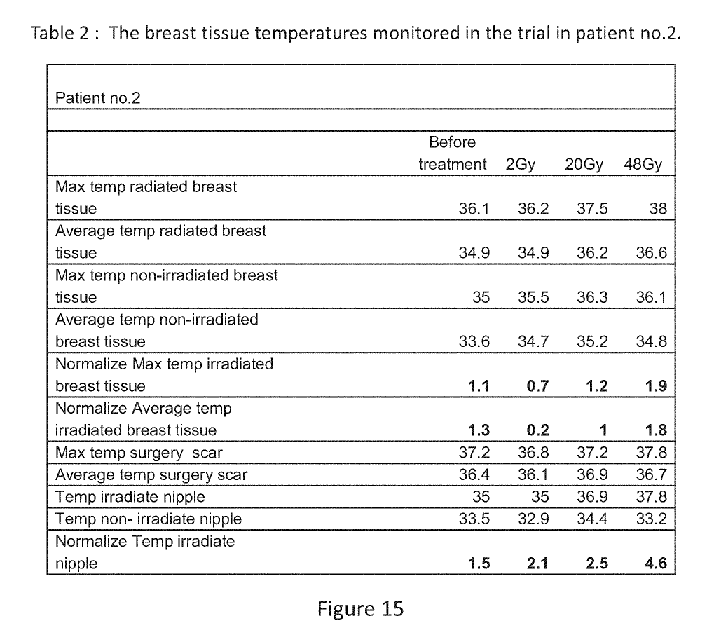

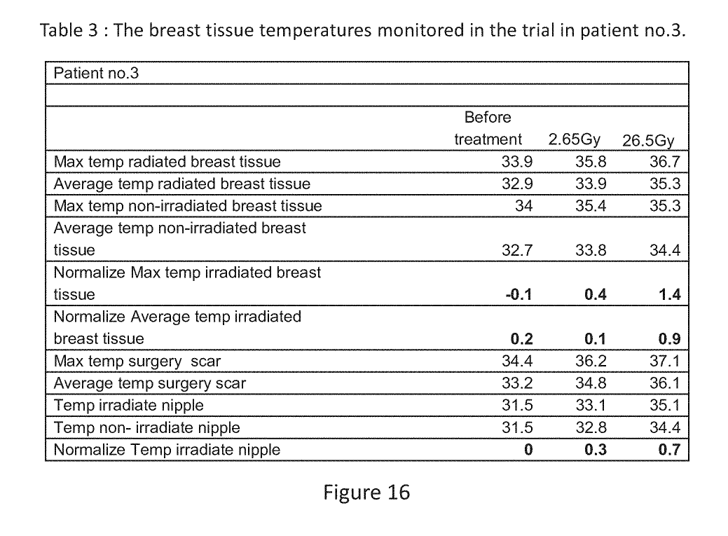

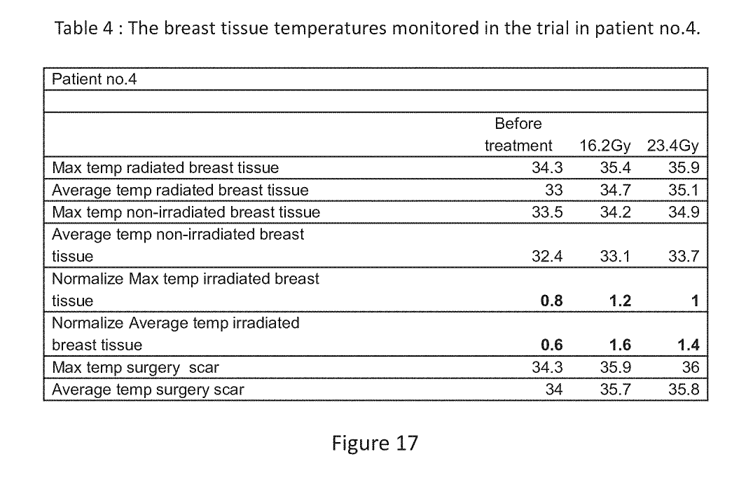

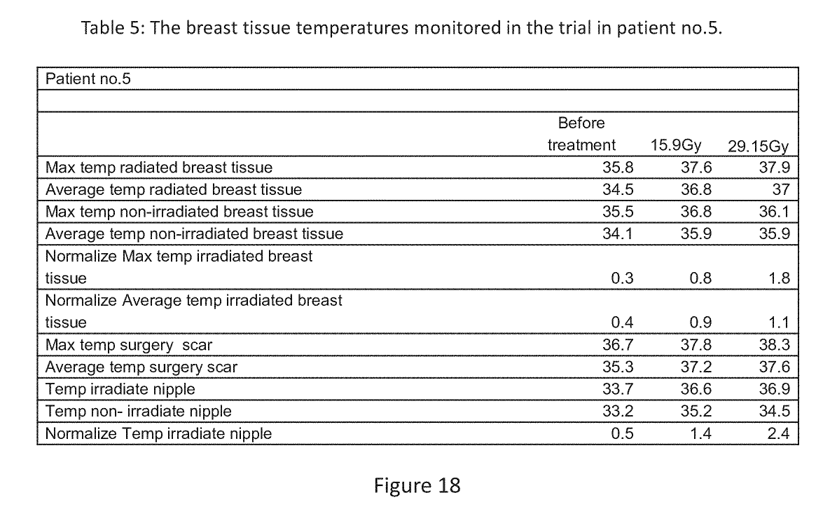

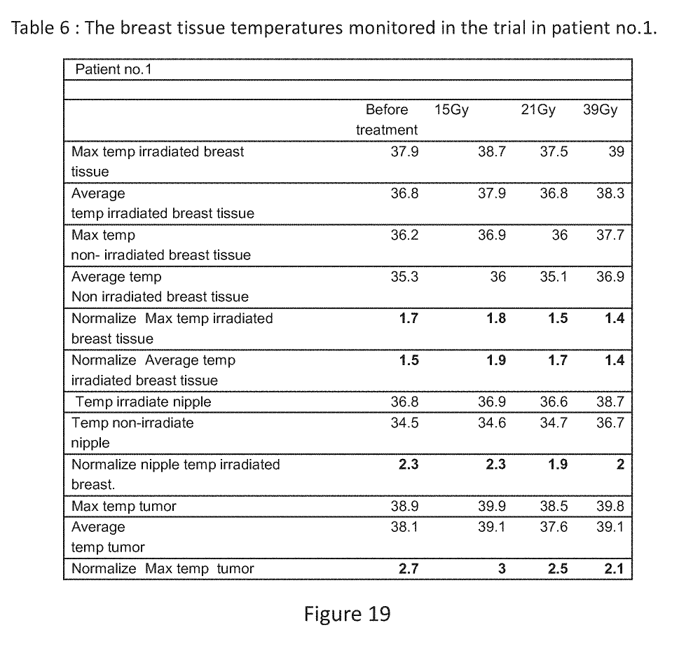

All patients were imaged prior to beginning radiotherapy, in accordance with some embodiments. Additional images of patient no. 2 were taken after 2, 20, and 48 Gy of radiotherapy. FIG. 3 shows the thermography of patient no. 2. The top image shows before irradiation, the middle image after a total dose of 20 Gy, and bottom image after a total dose of 48 Gy. The temperature scale in the image is 32-39° C. Additional images of patient no. 3 were taken after 2.65 and 26.5 Gy of radiotherapy. Patient no. 4 was imaged after 16.2 and 23.4 Gy of radiotherapy. Additional images of patient no. 5 were taken after 15.9 and 29 Gy of radiotherapy. In order to avoid environmental impact on the result, in accordance with some embodiments, a temperature of the radiated breast was normalized, according to some embodiments. A temperature of the non-irradiated breast was set as a reference temperature, according to some embodiments. Normalization of the irradiated breast temperature was calculated as a difference between the temperature of the irradiated and the temperature of the non-irradiated breast, in accordance with some embodiments. FIGS. 15-18 (Tables 2-5) present the maximal, average and normalized temperature of breast tissue in patients 2-5 as a function of the cumulative doses. In patient 4 the nipple cannot be detected. It is evident that each patient exhibited a rise in maximal and average temperatures of the irradiated breast. Patient no. 1 was additionally monitored after 15, 21 and 39 Gy of radiotherapy. FIG. 19 (Table 6) shows the maximal, average and normalized temperature in patent no. 1 as a function of the cumulative doses. For patient no. 1, the tumor area exhibited a rise in the maximal normalized temperature after a dose of 15 Gy, and drop in temperature after a dose of 21 and 39 Gy. The maximal and average temperatures of the irradiated breast and tumor dropped.

In some cases, as can be observed for example in FIG. 3, differences in the thermal distribution of non-targeted areas (e.g. the upper portion of the chest) may occur as a result of inflammation.

The Clinical Assessment of Radiotherapy for Patients in the Trial, in Accordance with Some Embodiments.

Patient 1, who was treated with palliative intent due to invasion of the skin by breast cancer, in accordance with some embodiments, exhibited good response during the radiotherapy period. She experienced a reduction in the tumor size, and after one month she was free of any clinical signs of the tumor in the treated breast.

Patients 2-5 that underwent radiotherapy as adjuvant treatment, in accordance with some embodiments, had no signs of disease in the breast one year after treatment.

### Discussion

In accordance with some embodiments, the main purpose of radiotherapy is to damage endothelial cells or vasculature and not tumor parenchymal cells [22, 23-27]. Apoptosis in tumor endothelial cells may lead to secondary death in tumor cells [22, 25]. Radiation-induced endothelial cell death may affect the efficacy of treatment [22, 25]. Some embodiments relate to assessing one or more changes in vasculature during radiotherapy, such as in blood vessels and/or capillaries leading to and/or surrounding and/or forming a part of a tumor. In the exemplary preliminary study described herein, vasculature changes were assessed using thermal imaging, in accordance with some embodiments. Some potential advantages of thermal imaging may include that is an available, non-irradiating, non-contact, and inexpensive technique.

In some cases, damage to the tumor's vasculature is the most important factor in the response to radiotherapy [23-27]. Apoptosis in tumor endothelial cells may lead to secondary death in tumor cells [22, 25]. The exemplary study described showed that the vascular changes that occur during treatment in the tumor area can be monitored by processing an image that highlights blood vessels with malignant properties, in accordance with some embodiments. Using thermography, in accordance with some embodiments, information about the efficacy of radiotherapy at a very early stage was obtained, optionally even before changes were evident in the size of the tumor. In some embodiments, methods and/or systems and/or devices as described herein may be used additional areas of the body, such as the head and neck.

In some cases, a decrease in the normalized temperature of the tumor area was observed. This may have occurred due to a reduction in the tumor's malignancy as a result of the radiotherapy [16, 17]. In some embodiments, the degree of tumor cooling provides an indication of the efficacy of the radiotherapy.

In some cases, the tissue temperature changes (e.g. decreases) as a function of the time that passed from a treatment session. Optionally, the tissue temperature is monitored at one or more times following a treatment session (e.g. irradiation session and/or chemotherapy session). In some cases, a temperature change in the tissue that is indicative of the tumor response to treatment is evident at, for example, 1 hour following a treatment session, 1 day following a treatment session, 1 week following a treatment session, 2 weeks following a treatment session, 1 month following a treatment session or intermediate, longer or shorter time periods. In some embodiments, thermal images of the tissue are acquired at one or more time points during and/or following treatment, for example at time points in which a change in the temperature due to the tumor's response to treatment is expected.

In some cases, a sharp rise in temperature as a result of the inflammatory process is exhibited. In some cases, the temperature rise stems from inflammation in the breast tissue, resulting from the irradiation [28]. The radiation induced damage to the DNA, which subsequently caused the activation of cytokines, potentially leading to inflammation and a rise in temperature [28]. In accordance with some cases, the higher the cumulative doses of radiation, the more severe the inflammatory process and the higher the temperature of the breast tissue.

In the study described, a difference was evident in the effect of the radiation on the temperature of a breast that underwent tumor resection as compared to a breast with a tumor. A possible reason for the decrease in the normalized temperature in the breast with the tumor may be due the tumor's reduced malignancy, which caused a reduction in the tumor's heat production capability [16, 17]. The reduction in the normalized temperature of the tumor area as a result of the reduced malignancy can attest the efficacy of the treatment. In some cases, the steeper the temperature drop, the more effective the treatment.

In some cases, the inflammatory process that leads to an increase in temperature in the entire breast masks the temperature changes in the tumor area. In accordance with some embodiments, an algorithm suitable to process the image of the blood vessels, making it possible to monitor vascular changes during treatment was developed. A potential advantage of thermography may include that it enables visualizing the physiology, in contrast to a CT or MRI for example, which are not only expensive and less readily available, but also show only the size of the tumor and not the physiological processes occurring before tumor size changes.

In all patients tested in this exemplary study, in the breast that underwent tumor resection the temperature of the surgery scar was higher than the temperature of the breast tissue, optionally as a result of inflammation subsequent to the surgery. In some embodiments, methods and/or devices as described herein are used for monitoring inflammation spread, level and/or effect on certain tissue types or regions, such as on scar tissue. In some cases, during radiotherapy, it was observed that the irradiated breast heated up as a result of inflammation [28].

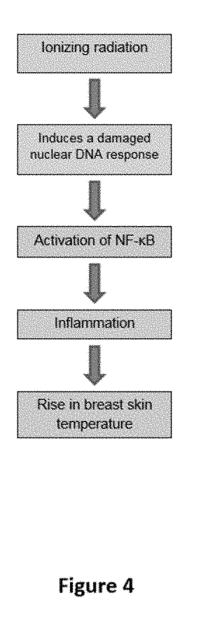

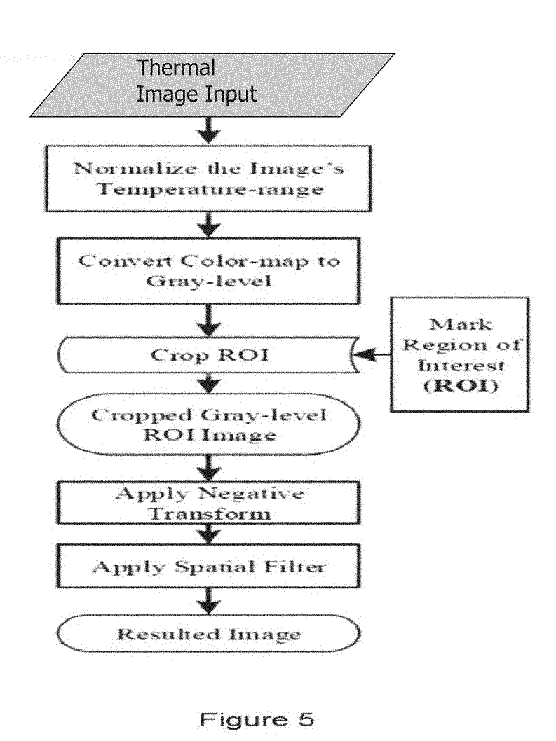

FIG. 4 shows a schematic description of the process that causes the temperature of the breast tissue to rise subsequent to radiotherapy. In some cases, radiotherapy causes damage to the DNA, which leads to the release of cytokines, resulting in an inflammatory process, which may cause the temperature of the breast tissue to rise [28].

In some embodiments, Thermography provides information about an inflammatory process that occurs in the irradiated area. In some cases, a large variety of classic or novel drugs may interfere with the inflammatory network in cancer and are considered to function as putative radiosensitizers. In some embodiments, thermal imaging can detect inflammation induced by radiotherapy. In some embodiments, targeting the signaling pathways caused by radiotherapy offers the opportunity to improve the clinical outcome of radiotherapy by enhancing radiosensitivity [28].

A method for monitoring cancer treatment is presented herewith, in accordance with some embodiments. In some embodiments, the method measures the physiological response to therapy, not only the structure of the tumor. Therefore, early in treatment, it may be possible to obtain information about the efficacy of the therapy. In addition, in accordance with some embodiments, thermography provides information about the inflammatory process that occurs in the tumor area, and controlling that inflammation may contribute to the efficacy of the treatment.

In some embodiments, assessing the efficacy of radiotherapy during the treatment makes it possible to change the treatment regimen, dose, and/or radiation field during therapy as well as to plan individualized treatment schedules for optimal treatment effectiveness, in accordance with some embodiments.

### REFERENCES

1. Nover A B, Jagtap S, Anjum W, Yegingil H, Shih W Y, Shih W, Brooks A D. Modern Breast Cancer Detection: A Technological Review. Int J Biomed Imaging 2009. doi: 10.1155/2009/902326
2. Therasse P, Arbuck S G, Eisenhauer E A, Wanders J, Kaplan R S, Rubinstein L, Verweij J, Van Glabbeke M, van Oosterom A T, Christian M C. New guidelines to evaluate the response to treatment in solid tumors. J Natl Cancer Inst 2000; 92: 205-16.
3. Agnelli J P, Barrea A, Turner C. Tumor location and parameter estimation by thermography. Math Comput Model 2011; 53: 1527-34.
4. Diakides M, Bronzino J D, Peterson D R. Medical Infrared Imaging—Principles and Practices, Boca Raton, Fla.: CRC Press, 2013.
5. Amalu W C, Hobbins W B, Head J F, Elliot RL. Infrared imaging of the breast—An overview. in Bronzino J D, Eds. The Biomedical Engineering Handbook. Medical Devices and Systems, Boca Raton, Fla.: CRC press, 2006; 25-1-25-20
6. Fok S, Eyk N, Tai K. Early detection and visualization of breast tumor with thermogram and neural network. J Mech Med Biol 2002; 2: 185-95.
7. Gautherie M. Thermobiological assessment of benign and malignant breast diseases. Am J Obstet Gynecol 1983; 147: 861-9.
8. Gautherie M, Kotewicz A Gueblez P. Accurate and objective evaluation of breast thermograms: Basic principles and new advances with special reference to an improved computer-assisted scoring system. in Thermal assessment of Breast Health New York: Springer, 1983; 72-97
9. Mital M, Scott E P. Thermal detection of embedded tumors using infrared imaging. J Biomech Eng 2007; 129: 33-9.
10. Ring E F J. Thermographic terminology. Acta Thermographica 1978; 2: 1-30.
11. Kennedy D A, Lee T, Seely D. A comparative review of thermography as a breast cancer screening technique. Integr Cancer Ther 2009; 8: 9-16.
12. Vaupel P, Kallinowski F, Okunieff P. Blood flow, oxygen and nutrient supply, and metabolic microenvironment of human tumors: a review. Cancer Res 1989; 49: 6449-65.
13. Macbeth R A, Bekesi J G. Oxygen consumption and anaerobic glycolysis of human malignant and normal tissue. Cancer Res 1962; 22: 244-8.
14. Kallinowski F, Schlenger K, Runkel S, Kloes M, Stohrer M, Okunieff P, Vaupel P. Blood flow, metabolism, cellular microenvironment, and growth rate of human tumor xenografts. Cancer Res 1989; 49: 3759-64.
15. Gautherie M, Gros C M. Breast thermography and cancer risk prediction. Cancer 1980; 45: 51-6.
16. Gautherie M. Thermopathology of breast cancer: measurement and analysis of in vivo temperature and blood flow. Ann New York Acad Sci 1980; 335: 383-415.
17. Yahara T, Koga T, Yoshida S, Nakagawa S, Deguchi H, Shirouzu K. Relationship between microvessel density and thermographic hot areas in breast cancer. Surg Today 2003; 33: 243-8.
18. Carmeliet P, Jain R K. Angiogenesis in cancer and other diseases. Nature 2000; 407: 249-57.
19. Jain R K. Molecular regulation of vessel maturation. Nat Med 2003; 9:685-93.
20. Reyes M, Dudek A, Jahagirdar B, Koodie L, Marker P H, Verfaillie C M. Origin of endothelial progenitors in human postnatal bone marrow. J Clin Invest 2002; 109: 337-46
21. Konerding M A, Miodonski A J, Lametschwandtner A. Microvascular corrosion casting in the study of tumor vascularity: a review. Scanning Microsc 1995; 9: 1233-44.
22. Park H J, Griffin R J, Hui S, Levitt S H, Song C W. Radiation-Induced Vascular Damage in Tumors: Implications of Vascular Damage in Ablative Hypofractionated Radiotherapy (SBRT and SRS). Radiat Res 2012; 177: 311-27.
23. Cramer W. Experimental observations on the therapeutic action of radium. Tenth Sci Rep Imp Cancer Res Fund 1932; 95-123
24. Denis F, Bougnoux P, Paon L, le Floch O, Tranquart F. Radiosensitivity of rat mammary tumors correlates with early vessel changes assessed by power Doppler sonography. J Ultrasound Med 2003; 22: 921-9
25. Garcia-Barros M, Paris F, Cordon-Cardo C, Lyden D, Rafii S, Haimovitz-Friedman A, et al. Tumor response to radiotherapy regulated by endothelial cell apoptosis. Science 2003; 300: 1155-9
26. Kolesnick R, Fuks Z. Radiation and ceramide-induced apoptosis. Oncogene 2003; 22: 2897-906.
27. Gulbins E, Kolesnick R. Raft ceramide in molecular medicine. Oncogene 2003; 22:7070-7.
28. Multhoff G, Radons J. Radiation, inflammation, and immune responses in cancer. Front Oncol. 2012 doi: 10.3389/fonc.2012.00058.

### Exemplary Tumor Detection and Staging Based on Thermography Results

According to some exemplary embodiments, thermography is used for early detection of tumors or other malignancies. In some embodiments, the thermal images acquired by thermography allows to detect blood vessel concentration. In some embodiments, the blood vessels indicate the presence of a pre-malignant or early stage malignant tumors. In some embodiments, the staging of a tumor, for example between a pre-malignant stage and an early malignant stage is performed using a combination of visible light imaging and thermal imaging.

Reference is now made to FIG. 6C depicting a process for detection and staging of a tumor using thermography, according to some embodiments of the invention.

According to some exemplary embodiments, thermography is performed at 620. In some embodiments, thermography is performed by taking one or more thermal images of a selected body or tissue area. In some embodiments, thermography is performed by placing a thermal imager configured for taking one or more thermal images outside the body. In some embodiments, the thermal imager is positioned outside the body to allow detection or monitoring of breast cancer. Alternatively or additionally, a thermal imager is inserted into the body, through a body orifice, for example to take thermal images of a selected region within the body. In some embodiments, the thermal imager is inserted into the body, for example through the vagina into at least part of the cervix, to allow detection and/or monitoring of cervical cancer.

In some embodiments, the thermal imager is inserted through the anus into the colon to allow detection and/or monitoring of GI tract associated cancers, for example colon cancer. In some embodiments, the thermal imager is inserted through the mouth, to allow detection and monitoring of oral cancer and/or laryngeal cancer.

In some embodiments, thermography is performed following a medical imaging process using a CT and/or a PET-CT and/or an MRI scan. Optionally, the medical imaging process is performed prior to thermography to allow focusing on a specific body region prior to thermography.

According to some exemplary embodiments, thermography is performed at 620 in combination with optical imaging. In some embodiments, optical imaging is used, for example to locate specific organs or tissues.

According to some exemplary embodiments, the one or more thermal images acquired at 620 are analysed at 622. In some embodiments, the analysis is performed using an algorithm for isolating features in the image to identify the tumor and/or the vasculature associated with the tumor. In some embodiments, the analysis results with a value indicative of the temperature and/or the entropy of the isolated features.

According to some exemplary embodiments, a tumor is detected at 624. In some embodiments, a tumor is detected based on the thermography analysis results. In some embodiments, the tumor is detected by identifying areas with large concentrations of blood vessels, which are optionally associated with a tumor. In some embodiments, these large concentrations of blood vessels produce excess of heat compared to other areas in the tissue. In some embodiments, areas of inflammation within the thermally scanned region are identified.

According to some exemplary embodiments, the tumor is classified at 626. In some embodiments, the tumor is classified as a malignant tumor or as a non-malignant tumor based on the thermography analysis results. In some embodiments, the tumor is classified as a malignant tumor or as a non-malignant tumor based the thermography analysis results and based on visible lights images. Optionally, the stage of the tumor is determined based on the thermography analysis results. In some embodiments, the tumor is classified as an advanced or as an early stage tumor.

According to some exemplary embodiments, a tumor is detected, classified and/or staged by comparing and matching the thermography analysis results to stored thermography analysis results or stored indications. In some embodiments, a tumor is detected, classified and/or staged using a machine learning algorithm stored in a memory.

According to some exemplary embodiments, the thermography analysis results indicate whether a tumor is present, the presence of an inflammatory process in the tissue and/or provide an indication regarding the stage or classification of the tumor.

According to some exemplary embodiments, a treatment protocol is selected at 628. I some embodiments, the treatment protocol is selected based on the tumor type, and/or the tumor stage determined at 624 and 626. In some embodiments, the selected treatment protocol comprises radiotherapy, brachytherapy, chemotherapy and/or immunotherapy.

According to some exemplary embodiments, at least one protocol parameter value is selected based on the tumor type, and/or tumor stage determined at 624 and 626.

According to some exemplary embodiments, cancer is treated at 630. In some embodiments, cancer is treated according to the treatment protocol and/or protocol parameters selected at 628.

In some embodiments, cancer is treated by radiotherapy by placing a radiation source outside the body or by inserting a radiation source through one of the body orifices. Alternatively or additionally, cancer is treated by brachytherapy, by placing a radiating implant inside the tumor tissue. In some embodiments, cancer is treated by chemotherapy and/or by immunotherapy. In some embodiments, cancer is treated at 630 by any combination of radiotherapy, brachytherapy, chemotherapy and/or immunotherapy.

According to some exemplary embodiments, cancer is treated at 630 by radiotherapy according to the selected treatment protocol at 628. In some embodiments, the radiotherapy treatment protocol comprises radiation intensity, radiating time per treatment session, the amount or radiation delivered to the tissue per treatment, per treatment session or per a selected time period, for example per day, per week or per month.

According to some exemplary embodiments, cancer is treated at 630 by brachytherapy, according to the selected treatment protocol at 628. In some embodiments, the treatment protocol comprises the number of radiating implant per an area or a volume of tumor tissue. In some embodiments, the treatment protocol comprises the radiation intensity per radiating implant. In some embodiments, the treatment protocol comprises the amount of radiation per treatment area or volume, optionally per a time period, for example per week, or per month.

According to some exemplary embodiments, cancer is treated at 630 by chemotherapy and/or immunotherapy according to the selected treatment protocol at 628, by one or more bioactive agents. In some embodiments, the treatment protocol comprises the composition of the bioactive agents. In some embodiments, the treatment protocol comprises the administration regime and/or dosage of the bioactive agents.

### Exemplary Modifying Cancer Treatment Based on Thermography Results

According to some exemplary embodiments, thermography of the tumor and/or tumor vasculature is used to determine if a treatment is efficacious. Optionally, following thermography a treatment protocol or at least one value of a treatment protocol parameter is modified.

Reference is now made to FIG. 6C depicting a process for determining treatment efficacy and optionally modifying a treatment based on thermography results, according to some embodiments of the invention.

According to some exemplary embodiments, cancer is treated at 630, as described above.

According to some exemplary embodiments, a one or more thermal images of the tumor area are acquired at 632, using thermography. In some embodiments, the thermal images of the tumor area are acquired before, after or between cancer treatment sessions. In some embodiments, the tumor area comprises the tumor and/or the tumor vasculature. In some embodiments, thermography is performed by placing a thermal imager configured for taking one or more thermal images of the tumor area, outside the body. In some embodiments, the thermal imager is positioned outside the body, for example to take one or more thermal images of a tumor area which is located near the outer surface of the body, for example a breast tumor. In some embodiments, the thermal imager is positioned outside the cervix to allow visualization of tumor that resides on or close to the cervix skin.

Alternatively or additionally, the thermal imager, is inserted into the body, through a body orifice as explained at 630, for example to take thermal images of a tumor located inside the body.

According to some exemplary embodiments, the one or more thermal images are analyzed at 634 as described at 622. In some embodiments, the analysis is performed before, after or between treatment sessions. In some embodiments, the analysis is performed using an algorithm to isolate features in the image of the tumor and/or the vasculature associated with the tumor. In some embodiments, the analysis results with a value indicative of the temperature and/or the entropy of tumor and/or the vasculature associated with the tumor.

According to some exemplary embodiments, the analysis comprises monitoring the change in temperature of the tumor and/or the vasculature associated with tumor during the treatment. In some embodiments, reduction in temperature values as the treatment progresses is an indication of an efficacious treatment. In some embodiments, reduction of at least 2% of the initial temperature measured prior to treatment, for example 2, 3, 4, 5% or any intermediate or larger value is an indication of an efficacious treatment. In some embodiments, reduction in temperature values of the tissue at the tumor area to the temperature of a non-tumorigenic tissue or close to that is an indication of an efficacious treatment. Alternatively, stable or increasing temperature values measured as the treatment progresses are indicative of a non or less efficacious treatment. In some embodiments, stable or increasing temperature values indicate an inflammation process in the analyzed area.

According to some exemplary embodiments, the treatment efficacy is determined at 636. In some embodiments, the treatment efficacy is determined based on the analysis performed at 634. In some embodiments, the treatment efficacy is determined by comparing one or more thermal images taken before and/or during the treatment. In some embodiments, the treatment efficacy is determined by comparing the signal values of the tumor and/or the vasculature before and after the treatment. Optionally, the treatment efficacy is determined by comparing the analysis results of thermal images taken before and/or during the treatment.

According to some exemplary embodiments, the treatment is modified at 638. In some embodiments, the selected treatment protocol is modified. In some embodiments, the treatment is modified if the treatment efficacy, optionally as determined at 634 is not a desired efficacy. Alternatively, at least one treatment parameter, for example one or more of the treatment parameters described at 630, is modified at 634.

According to some exemplary embodiments, if the efficacy of a radiotherapy treatment is not a desired efficacy, then the radiotherapy treatment is modified at 638 by increasing the radiation dose delivered to the tumor and/or the radiation duration. Alternatively, chemotherapeutic agents that optionally act as radio-sensitizers are added to the radiotherapy treatment. In some embodiments, if the efficacy of a radiotherapy treatment at 630 is not a desired efficacy as determined at 636, then the radiotherapy is replaced by an alternative treatment, for example a chemotherapy treatment and/or an immunotherapy treatment.

According to some exemplary embodiments, if the efficacy of a chemotherapy or an immunotherapy treatment is not a desired efficacy as determined at 636, then a different dosage regime is selected. Alternatively, a different drug or a different combination of drugs is selected. In some embodiments, if the efficacy of a chemotherapy or an immunotherapy treatment is not a desired efficacy then an alternative treatment is selected, for example a radiotherapy treatment, a brachytherapy treatment, an immunotherapy treatment or a chemotherapy treatment is selected. Alternatively, the chemotherapy or immunotherapy treatment is combined with one or more of the alternative treatments listed above.

Reference is now made to FIG. 6D depicting a process for tumor characterization following treatment based on thermography, according to some embodiments of the invention.

According to some exemplary embodiments, the tumor is classified following treatment at 640. In some embodiments, the tumor is classified between a non-malignant, a malignant or a metastatic tumor based on the thermography analysis results performed at 634. Alternatively, the cancer stage is determined based on the thermography analysis results performed at 634. In some embodiments, the tumor is classified as described at 626. In some embodiments, the tumor is classified based on the analyzed measured thermal profile of the tumor and/or vasculature associated with the tumor during the treatment. In some embodiments, the measured thermal profile is compared to thermal profiles or indications of thermal profiles stored in a memory. In some embodiments, the comparison allows to match a stored thermal profile or a stored indication associated with a determined tumor type and/or tumor stage to the measured thermal profile.

According to some exemplary embodiments, a tumor profile is determined at 642 based on thermography, according to some embodiments of the invention. In some embodiments, the tumor resistance or sensitivity to the treatment provided at 630 is determined.

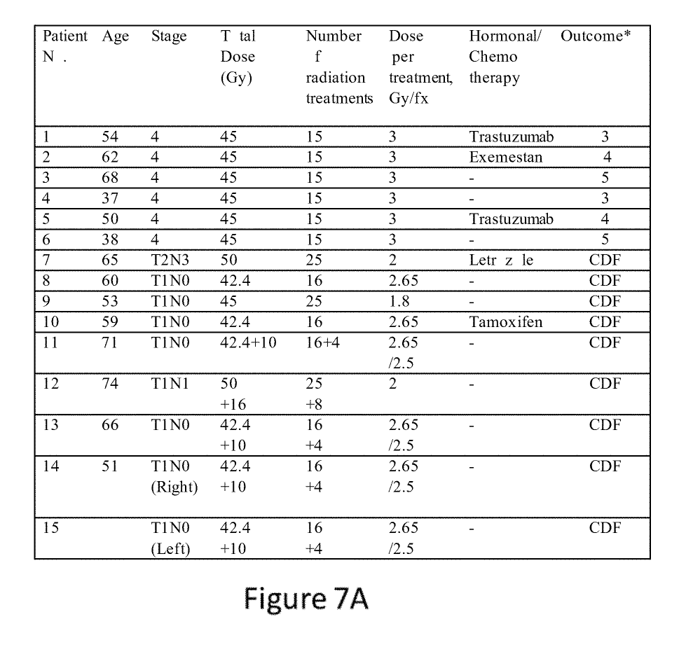

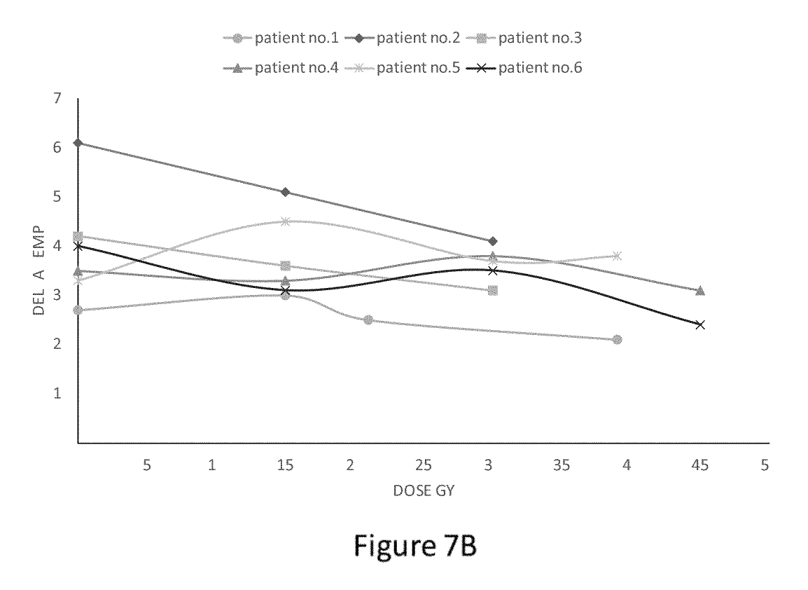

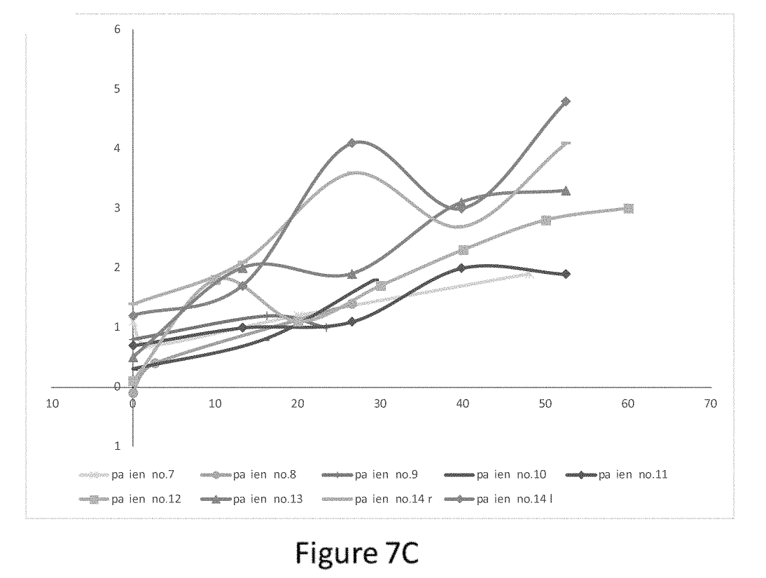

According to some exemplary embodiments, an inflammation process is detected at 644. In some embodiments, an inflammation process in the tissue is detected when the temperature of the tumor and/or the associated vasculature is not reduced or increases following treatment. Optionally, when the temperature is not decreased or increases the risk of developing radiation recall dermatitis increases, as described in FIG. 7B.

According to some exemplary embodiments, the treatment is modified at 638 according to the tumor staging determined at 640 and/or according to the tumor profile determined at 642 or according to the inflammation detected at 644. In some embodiments, if a risk for developing radiation recall dermatitis is detected, then the treatment is optionally modified by selecting a specific chemotherapeutic drug or a specific mix of drugs.

### Exemplary Monitoring Cancer Treatment Based on Tissue Temperature

According to some exemplary embodiments, breast cancer patients, for example 6 stage-IV breast cancer patients and 8 patients (9 breasts) who underwent tumor resection, are monitored by a thermal camera prior to radiotherapy sessions over several weeks of treatment. In some embodiments, the thermal images taken during radiotherapy are analyzed and the maximal temperatures of the breast tissue are calculated, optionally compared to the actual side effects. In some embodiments, in patients with active tumors, the images of the breast obtained during radiotherapy treatment are processed by an algorithm that highlights blood vessels with malignant properties.

Reference is now made to FIG. 7A, depicting a summarizing table of patients information, according to some embodiments of the invention.

According to some embodiments, breast skin temperature is monitored in breast cancer patients, for example 14 women (15 breasts), by thermography before and/or during radiotherapy. In some embodiments, patients underwent CT simulation for 3D treatment planning. In some embodiments, 6 patients (numbers 1-6, FIG. 7A) had stage IV breast cancer and viable tumor in the breast. In some embodiments, these patients are intended to receive 39-45 Gy, optionally divided into 13-15 fractions of 3 Gy/fraction. In some embodiments, the radiotherapy therapy treatment is administered 5 days a week, for 2.5-3 weeks in total.

According to some exemplary embodiments, 8 patients (9 breasts) (numbers 7-14, FIG. 7A) underwent tumor resection and received radiotherapy as adjuvant treatment. Treatment data (radiotherapy, chemotherapy, and hormonal therapy) are summarized in FIG. 7A.

According to some exemplary embodiments, the patients are monitored throughout the period of radiotherapy by a thermal camera with images of the breasts optionally taken regularly before radiotherapy treatment sessions. In some embodiments, to maintain fixed environmental conditions, the room temperature was set to 24-26° C. and the room humidity to 50-57%. Additionally, fluorescent lamps were turned off during image acquisition. In some embodiments, the thermal images are analyzed using an analysis software, for example the FLIR Tool software (ResearchIR), which optionally calculates the maximal temperatures of the breast tissue. In some embodiments, to avoid environmental impact on the results, the radiated breast temperature is normalized. Optionally, the radiated breast temperature is normalized to a non-irradiated area which is set as a reference temperature; the same area size is always taken as a reference.

According to some exemplary embodiments, for example in patients 1-6 with active tumors, the images of the breast obtained during radiotherapy treatments are processed by a processing software, for example MATLAB software. In some embodiments, to attain a quantified measure of the change that occurred in vasculature in the process of thermal imaging during radiotherapy, a value of the image entropy is calculated. Entropy is a statistical measure of randomness that can be used to characterize the texture of an input image. In some embodiments, entropy characterizes the homogeneity of the image, for example the higher homogeneity -the lower is the entropy value.

According to some exemplary embodiments, the concentration of blood vessels affects the homogeneity of the thermal image. In some embodiments, the higher the concentration of blood vessels, the lower homogeneity. Therefore, in some embodiments, the measure of entropy is used to evaluate the change in the vasculature.

According to some exemplary embodiments, the calculated entropy values are subjected for statistical analysis using statistical software, for example Statistical Package for Social Sciences (SPSS) software. In some embodiments, the statistical analysis comprises analysis of variance with repeated measures for breast temperature measurements. Additionally, nonparametric Spearman's rank-order correlations is used to examine possible correlation between reduction in vasculature in the process of thermal imaging and clinical outcome.

According to some exemplary embodiments, for example as shown in FIG. 7A, patients 1-6 are all stage IV disease and receive radiation for palliation due to viable breast tumor, with either skin invasion, ulceration or painful breast mass. In some embodiments, patients 7-14 receive adjuvant radiation following surgery, with no viable tumor, with patient number 14 receives bilateral radiation treatment due to bilateral breast tumor.

Reference is now made to FIG. 7B, depicting the maximal normalized temperature of patients 1-6, who had active tumors, as a function of the cumulative radiation dose. According to some exemplary embodiments, except for patient no. 1, all had negative slope with decrease in the delta temperature when compared to the contralateral untreated breast during radiation.

Reference is now made to FIG. 7C depicting the maximal normalized temperature of breast tissue in the patients 7-14 (9 breasts) who underwent radiotherapy as adjuvant treatment, as a function of the cumulative radiation dose, according to some embodiments of the invention. In some embodiments, for example as shown in FIG. 7C, patients who underwent radiotherapy as adjuvant treatment exhibited a rise in maximal temperature of the whole breast; this difference compared to the group of patients who had active tumors, as shown in FIG. 7B is statistically significant (P=0.001).

### Breast Temperature Measurements

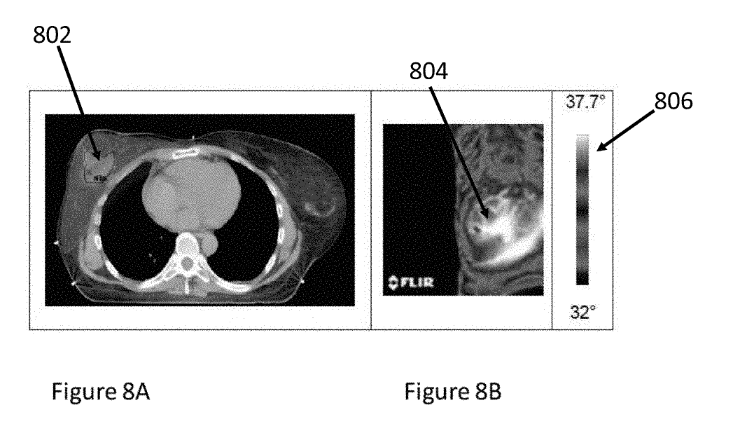

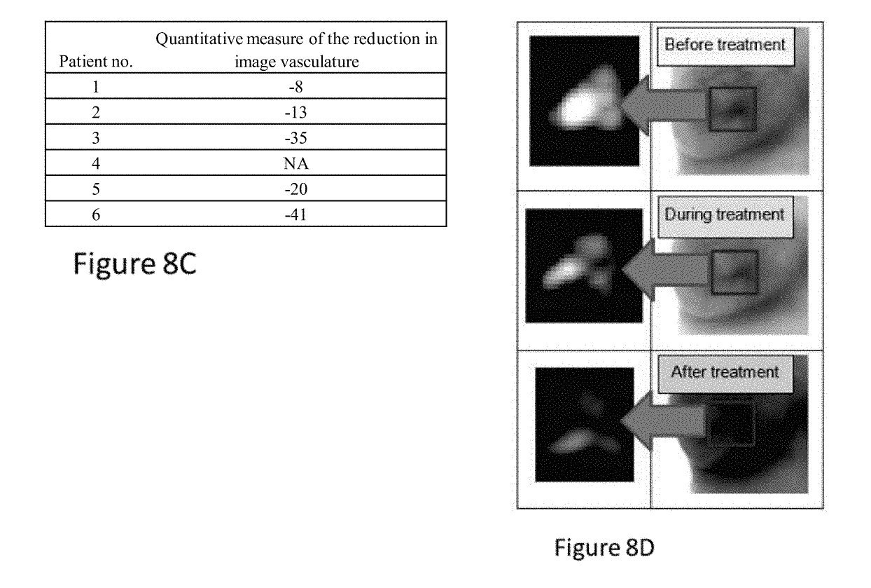

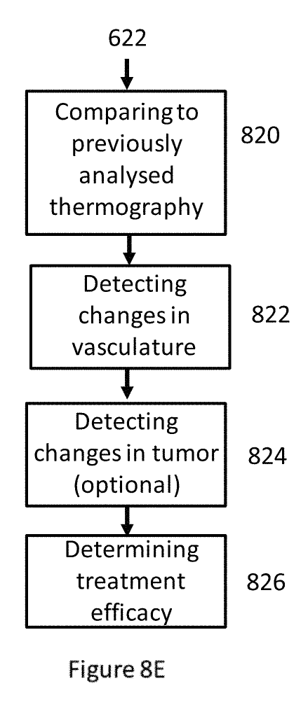

Reference is now made to FIGS. 8A and 8B depicting images obtained from a patient with a breast tumor, according to some embodiments of the invention.

According to some exemplary embodiments, for example as shown in FIG. 8A, a CT scan is taken to identify a tumor 802 inside a breast. Optionally, the tumor 802 volume is contoured on the CT scan taken prior to radiotherapy. According to some exemplary embodiments, for example as shown in FIG. 8B, a thermal image taken at the same time or within a short time interval. In some embodiments, the hot area 804 on the skin, as indicated by color scale 806, correlates with the shape of the tumor on the CT.

### Image Pocessing

According to some exemplary embodiments, the thermal images of cancer patients, for example patients 1-6, which were taken before treatment and after 21-30Gy, are processed by an algorithm that highlights blood vessels. In some embodiments, for example as shown in FIG. 8C, there is a visual reduction in the vasculature and quantitative reduction in vasculature. In some embodiments, the changes in the percentage of entropy for patients (1-6), is calculated by comparing the baseline image before treatment and the image after 30 Gy. In some embodiments, the entropy value is a quantitative measure of the reduction in image vasculature. In some embodiments, for example if the tumors are skin invading with ulcerative masses, the algorithm cannot detect vasculature, as in the case of patient 4.

Reference is now made to FIG. 8D, depicting thermal images of a patient's breast cancer taken before, during and after treatment, according to some exemplary embodiments of the invention. According to some exemplary embodiments, thermal images of a cancer patient, for example patient 1, are taken before (0 Gy), during (21 Gy), and at the end of radiation treatment (39 Gy). In some embodiments, the left-hand panel shows the tumor area, highlighted by the red box in the main image, after image processing. In some embodiments, for example in the thermal image at the top, taken prior to the beginning of treatment, after image processing, the concentration of blood vessels with malignant properties is apparent. In some embodiments, for example as shown in the middle thermal image, taken during radiotherapy (21 Gy), the vasculature is visibly reduced. In some embodiments, for example as shown in the thermal image at the bottom, taken at the end of treatment (after 39 Gy), a sharp decrease in the concentration of blood vessels with malignant properties is evident.

Reference is now made to FIG. 8E depicting a process for detecting changes in vasculature, according to some embodiments of the invention. In some embodiments, the process follows the changes in vasculature and tumor following treatment as shown in FIG. 8D. According to some exemplary embodiments, the analysed thermography images at 620 shown in FIG. 6C are compared to previously analysed thermography images at 820. Optionally, the previously acquired thermography images are taken prior to a treatment. In some embodiments, the previously analysed thermography images or indication for such images are stored in a memory. According to some exemplary embodiments, changes in vasculature between the two or more images are detected at 822. Additionally or alternatively, changes in the tumor are detected at 824, based on the comparison between the two or more images. According to some exemplary embodiments, the efficacy of a treatment is determined at 826. In some embodiments, the efficacy of the treatment is determined based on the changes in vasculature between the images that were acquired after the treatment to the images acquired prior to the treatment. In some embodiments, a treatment is efficacious is a decrease in the detected vasculature in identified following the treatment.

### Clinical Otcome

According to some exemplary embodiments, the clinical outcomes of cancer patients, for example patients 1-6 is assessed by physicians according to a scale from 1 to 5: Grade 1: no improvement; Grade 2: slight decrease in tumor mass; Grade 3: moderate decrease in tumor mass; Grade 4: considerable decrease in tumor mass; 5: extreme decrease in tumor mass. In some embodiments, patient who underwent radiotherapy as an adjuvant treatment, for example Patients 7-14, are characterized as clinically disease free (CDF). Reference is now made to the table in FIG. 7A, which depicts the clinical outcome for each patient, according to some embodiments of the invention. In some embodiments, the Spearman's Rho correlation showed statistically significant correlation between the reduction in vasculature and clinical outcome (P=0.01385, R=0.94868). In some embodiments, the highest the vasculature reduction seen during treatment, the better clinical response detected. Results

According to some exemplary embodiments, patients with active tumors exhibited drops in maximal temperature. In some embodiments, the cooling occurred due to a reduction in the tumor vasculature and/or necrosis, optionally as a result of the radiotherapy.

According to some exemplary embodiments, patients who underwent radiotherapy as adjuvant treatment exhibited a rise in maximal temperature. In some embodiments, the rise in temperature results from inflammation in the breast tissue due to the irradiation. This difference between the groups is statistically significant (P=0.001). In some embodiments, the vascular changes that occur during treatment in the tumor area are monitored by the processed image that shows blood vessels with malignant properties. In some embodiments, a quantitative measure of the reduction of vasculature is generated and a statistically significant correlation is observed between reduction in vasculature and clinical outcome (P=0.01385, R=0.94868).

### Discussion

According to some exemplary embodiments, thermal imaging is used to create a direct correlation between tumor vasculature reduction during radiation and the clinical response of the tumor to radiation treatment. In some embodiments, as described in FIG. 7C a significant elevated skin temperature during radiotherapy is measured in women with no active tumor in the breast in contrast with the temperature reduction in breasts with active tumor responding to the radiation.

In Some embodiments, tumor vasculature is essential for keeping the tumor alive and facilitating its growth and viability. Optionally, solid tumors must create neo-angiogenesis at a size of 1-2 mm to avoid necrosis. In some embodiments, the newly formed blood vessels develop abnormally, they dilate and become tortuous while retaining their capillary-like structure with no further differentiation for arteries are venules. Alternatively or additionally, cancer cells in the tumor form de-novo vascular network, induced by hypoxia.

In some embodiments, during radiotherapy, the normalized temperature of breasts with tumors decreased, and the temperature of breasts without tumors increased (P=0.001), for example as shown in FIGS. 7B and 7C respectively. Optionally, the cooling apparently occurred due to a reduction in the tumor's vasculature, hence reducing its aggressiveness, as a result of the radiotherapy. In some embodiments, The rise in temperature in adjuvant cases stems from the inflammation process in the healthy breast tissue resulting from the radiation. Radiation-induced damage to DNA causes activation of cytokines, vascular dilatation of health vessels and leakage and leads to inflammation process and to a rise in temperature.

In some embodiments, the inflammatory process that leads to an increase in temperature in the entire breast masks changes in temperature in the tumor area. In some embodiments, to enhance blood vessels, an algorithm that highlights the blood vessels is used. Optionally, the enhancement of blood vessels in the processed image enables monitoring of vascular changes during treatment.

### Exemplary Predicting Radiation Recall Dermatitis

According to some exemplary embodiments, radiation recall dermatitis is an acute inflammatory reaction confined to previously irradiated areas that can be triggered when chemotherapy agents are administered after radiotherapy. In some embodiments, monitoring an increase in temperature, for example breast skin temperature during radiation therapy predicts the development of radiation recall dermatitis. In some embodiments, the increase is temperature is detected in an area of the breast skin which is located near the tumor region. Optionally the breast skin is located in a distance that is shorter than 50 mm from the closest tumor tissue, for example 50, 40, 10, 5, 2 or any intermediate or lower distance.

Reference is now made to FIG. 7B depicting a thermal profile of patients during radiotherapy, according to some embodiments of the invention. According to some exemplary embodiments, when monitoring tissue treatment during radiotherapy, some patients, for example patient No. 5 show a rising gradient at the beginning of the radiation treatment compared to the reference or baseline temperature. In some embodiments, the rise in temperature is detected after a 5 to 25 Gy cumulative dose, for example after 5, 10, 15 Gy cumulative dose or any intermediate or larger value.

According to some exemplary embodiments, in these patients, for example patient No. 5, the temperature of the tumor declines but still remains higher than the baseline temperature obtained before the first radiation session. Additionally, the normalized temperature at the end of the radiation treatment is higher for patient No. 5 compared to the rest of the patients.

According to some exemplary embodiments, patients who underwent radiotherapy develop radiation recall dermatitis following radiotherapy. Optionally, these patients develop radiation recall dermatitis after they receive chemotherapy, for example as in the case of patient No. 5. In some embodiments, a Pearson correlation coefficient is used for analyzing the correlation between the temperature gradient to the recall radiation phenomenon outbreak. In patient No. 5 the calculated Pearson correlation coefficient is r=0.8657, which demonstrates a strong positive correlation between the recall burst and the temperature gradient.

According to some exemplary embodiments, detecting an increase in temperature of the tissue allows, for example to adjust or modify the chemotherapy treatment following radiotherapy. Optionally, the chemotherapy treatment is modified or selected based on the prediction to develop radiation recall dermatitis. In some embodiments, larger time interval between treatment modalities will be applied in patients predicted to develop radiation recall dermatitis. Alternatively or additionally, the dose of the anti-cancer agent is reduced in these patients.

According to some exemplary embodiments, there are at least two processes affecting tissue temperature: 1. the rise in temperature following radiotherapy due to an inflammatory process and 2. the cooling down of the tumor tissue resulting from reduced tumor viability. In some embodiments, for example when an inflammation process is developed, the cooling of the tissue is attenuated or has a reduced effect on the overall temperature of the tissue. In some embodiments, for example as in patient No. 5 the heating of the tumor area was so radical that the cooling effect, resulting from the destruction of tumor blood vessels, was indistinguishable when analyzing the normalized temperature measurements. Although the tumor shrunk, the inflammatory reaction was the most dominant process spotted in the thermographic imaging.

### Exemplary Monitoring Brachytherapy Using Thermal Imaging

According to some exemplary embodiments, brachytherapy which is a radiotherapy based on radioactive implants is monitored using thermal imaging. In some embodiments, brachytherapy is used to treat cervical cancer, endometrial cancer, intraoperative application for intra abdominal sarcoma, and head and neck cancer. In some embodiments, brachytherapy is monitored from within the body, optionally by inserting a probe for thermal imaging into the body. In some embodiments, for example, to monitor tumor response in cervical cancer or in endometrial cancer, the camera is inserted into the vagina, to capture the temperature of the cervical or endometrium area, respectively.

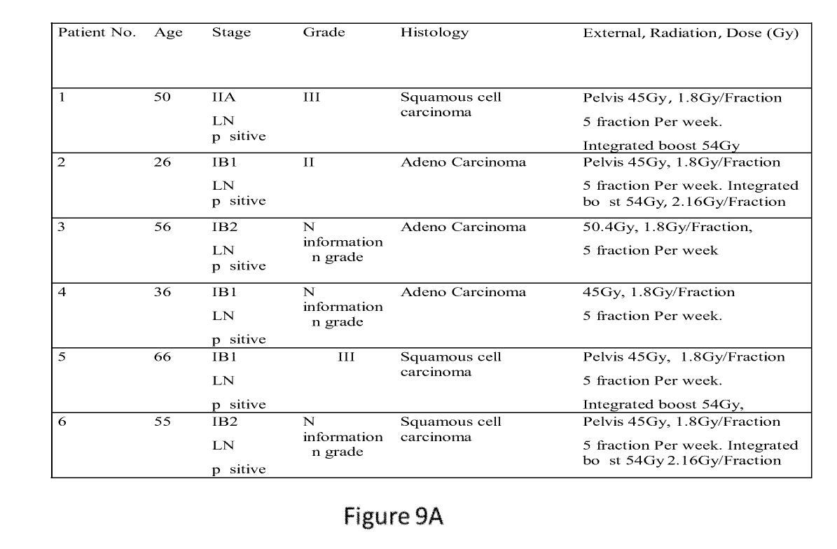

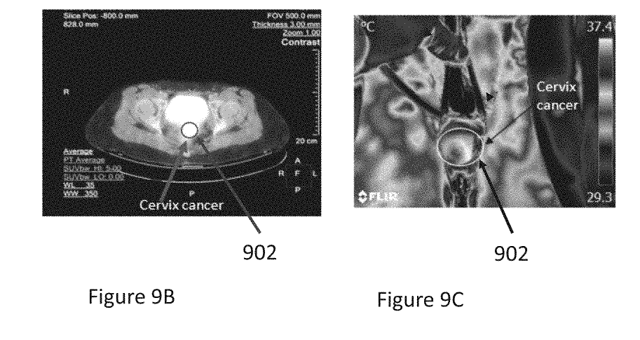

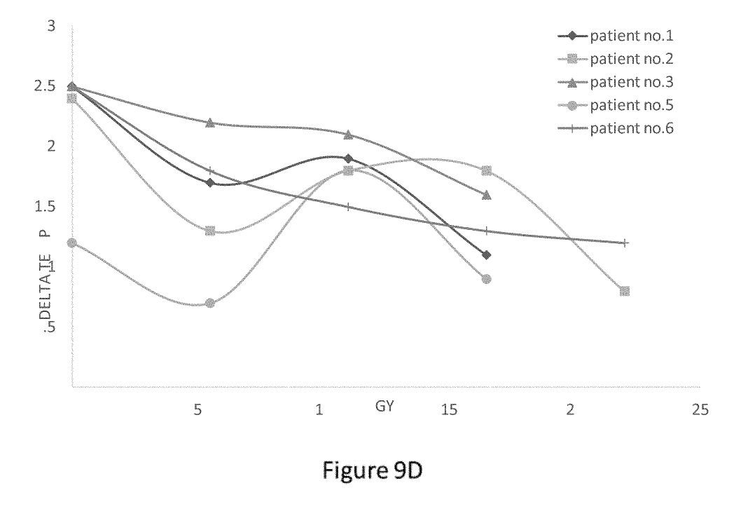

Reference is now made to FIG. 9A depicting a table summarizing the details of 6 patients that underwent brachytherapy, according to some embodiments of the invention.

According to some exemplary embodiments, 6 patients received brachytherapy for advanced cervical carcinoma. In some embodiments, the age of the patients, histopathologic diagnoses, histologic grade, clinical stage, treatment, and outcome are summarized in FIG. 9A. In some embodiments, for women who develop locally advanced cervical cancer, the standard of care combined EBRT plus brachytherapy with optionally concurrent chemotherapy. In some embodiments, as shown in the table in FIG. 9A, subjects received IMRT external radiotherapy given as 1.8-Gy daily fractions, 5 days/week and addition brachytherapy 5 fraction 5.5 Gy for fraction chemotherapy Carboplatin or Cisplatin 35 mg/m² #5 the total dose external dose varied from 50 to 65 Gy for external radiation and 27.5 for brachytherapy radiation.

Reference is now made to FIGS. 9B and 9C depicting images of the cervix taken before brachytherapy treatment, according to some embodiments of the invention. According to some exemplary embodiments, for example as shown in FIG. 9B, a PET scan of the cervix region is taken prior to brachytherapy treatment. Alternatively, other imaging techniques can be used, for example CT, MRI imaging techniques. Optionally, these techniques are used to identify the tumorigenic tissue region within the cervix. According to some embodiments, a thermal imaging image is taken during or after the PET scan, for example as shown in FIG. 9C. In some embodiments, the thermal imaging is performed by placing a thermal imager outside the body. Alternatively, the thermal imager is inserted into the vagina that is opened by a speculum, which is the normal, accepted way to perform gynecological exam, does not hurt, and enables direct vision to the cervix uteri.

Reference is now made to FIG. 9D depicting the change in the delta temperature values between the maximal temperature and the minimal temperature of the cervix during brachytherapy in patient 1-6 vs brachytherapy total dose, according to some embodiments of the invention. According to some exemplary embodiments, the maximal temperature is measured within the boundaries of the tumor tissue in the cervix. In some embodiments, the minimal temperature is measured outside the boundaries of the tumor tissue, in the same cervix of the same patient.

According to some exemplary embodiments, for example as shown in FIG. 9D, the delta temperature is reduced as the brachytherapy dose increases. In some embodiments, the reduction in the delta temp occurred due to a reduction in the tumor's aggressiveness, as a result of the brachytherapy. In some embodiments, the delta temperature increases between dosages 5 Gy to 17 Gy, compared to the delta temperature in dosages between 0 to 5 Gy. In some embodiments, the rise in temperature stems from inflammation in the cervix tissue resulting from the irradiation. In some embodiments, radiation-induced damage to DNA causes activation of cytokines, and leads to inflammation and to a rise in temperature.

### Exemplary Algorithm

According to some exemplary embodiments, an algorithm is used for tumor detection using thermal imaging. Additionally or alternatively, the algorithm is used to produce a quantified estimation of a tumor reduction and/or reduction of the tumor's vasculature during radiotherapy.

According to some exemplary embodiments, thermal images of a tumor tissue are taken prior to and/or during a radiotherapy treatment. In some embodiments, the thermal images are processed by an analysis software, for example MATLAB software. In some embodiments, the metabolic activity of a tumor is abnormal when compared to the metabolic activity of a normal healthy tissue. The higher the tumor malignancy, the more heat it produces. Therefore, a change in tumor area temperature during radiotherapy treatment is optionally a measure of the tumor's response to the treatment. In some embodiments, the algorithm is used to filter the tumor from the original image and evaluate the changes occurring during radiotherapy.

Reference is now made to FIG. 10A depicting the main steps of an algorithm for analysis of thermal images, according to some embodiments of the invention.

According to some exemplary embodiments, the algorithm consists of four main steps: (1) preprocessing 1002, (2) tumor and vasculature detection and monitoring 1004, (3) feature extraction 1006, and (4) generating a quantitative measure of treatment efficacy 1004.

In some embodiments, preprocessing 1002 comprises converting the image into gray scale, normalizing the image matrix, and determining a region of interest (ROI). In some embodiments, at 1004 a filter to highlight the tumor and the vasculature is used in the second step. In some embodiments, the filter is initially used to show vessels in angiography imaging, which optionally have high contrast. Optionally at 1006 the filter is used to identify blood vessels (long and/or narrow hot objects). Alternatively or additionally, the filter is used to identify blobs of heat which are optionally an indication of a malignant tumor, from the thermal image.

According to some exemplary embodiments, entropy is a statistical measure of randomness that can be used to characterize the texture of the input image. In some embodiments, at feature extraction stage to attain a quantified measure of texture changes during radiotherapy, the entropy of the crop tumor area is calculated using the following entropy calculation equation:

H(x)=−Σₖ₌₁ⁿp(xₖ) log₂ p(xₖ)

In some embodiments, the probability density p(xₖ) is needed for calculating the image entropy. Optionally, this parameter is being estimated using a gray scale histogram.

In some embodiments, a score of the efficacy of the treatment is generated at 1008. Figure shows the structure of the presented method. Details of each section of the proposed algorithm will be presented.

### Exemplary Preprocessing

Reference is now made to FIG. 10B, depicting preprocessing of a thermal image, for example preprocessing 1002 according to some embodiments of the invention.

According to some exemplary embodiments, a thermal imaging color image 1012 is converted into a gray scale image 1014. In some embodiments, a fixed temperature range between 4-10° C., for example 4, 5, 7° C. or any intermediate temperature is set in all images. In some embodiments, in the fixed temperature range, for example the 7° C. range, physiological changes in human tissue are identified. Optionally, a fixed temperature range allows to compare between the entropy of the images. In some embodiments, the identified tumor area 1016 is cropped, for example to focus on the changes occurring in the tumor area during radiotherapy.

### Exemplary Tumor Detection and Monitoring

According to some exemplary embodiments, the gray value around a point is described by a two-dimensional Taylor series whose center is in the same point. Let us look at a general two-dimensional Taylor series:

$$f ( x , y ) = f ( {x}_{0} , {y}_{0} ) + {f}_{x} ( {x}_{0} , {y}_{0} ) \cdot ( x - {x}_{0} ) + {f}_{y} ( {x}_{0} {y}_{0} ) \cdot ( y - {y}_{0} ) ++ \frac{1}{2} \cdot {f}_{xx} ( {x}_{0} , {y}_{0} ) \cdot {( x - {x}_{0} )}^{2} + {f}_{xy} ( {x}_{0} , {y}_{o} ) \cdot ( x - {x}_{0} ) \cdot ( y - {y}_{0} ) + \frac{1}{2} {f}_{yy} ( {x}_{0} {y}_{0} ) \cdot {( y - {y}_{0} )}^{2}$$

$$\vec{∇} f = ( {f}_{x} , {f}_{y} ) , Δ \vec{x} = ( x - {x}_{0} , y - {y}_{0} ) , H = ( \begin{matrix}{f}_{xx} & {f}_{xy} \\ {f}_{yx} & {f}_{yy}\end{matrix} ) \\ {f}_{xy} = {f}_{yx}$$

it is can be seen that:

$$\begin{matrix}Δ \vec{x} H Δ \vec{x} = ( x - {x}_{0} , y - {y}_{0} ) ( \begin{matrix}{f}_{xx} & {f}_{xy} \\ {f}_{xy} & {f}_{yy}\end{matrix} ) ( \begin{matrix}x - {x}_{0} \\ y - {y}_{0}\end{matrix} ) \\ == ( {f}_{xx} ( x - {x}_{0} ) + {f}_{xy} ( y - {y}_{0} ) , {f}_{xy} ( x - {x}_{0} ) + {f}_{yy} ( y - {y}_{0} ) ) \\ ( \begin{matrix}x - {x}_{0} \\ y - {y}_{0}\end{matrix} ) \\ == {f}_{xx} \cdot {( x - {x}_{0} )}^{2} + 2 \cdot {f}_{xy} \cdot ( x - {x}_{0} ) \cdot ( y - {y}_{0} ) + {f}_{yy} \cdot {( y - {y}_{0} )}^{2}\end{matrix}$$

I can now write:

f(x,y)=f(x₀,y₉)+\vec{∇}f·Δ\vec{x}+½·∇\vec{x}H Δ\vec{x}

In some embodiments, when we are at the center of an object that resembles a “hole” (a dark area on a light background), the first derivative approximates to zero since we are in the bottom area of that hole. In some embodiments, if we want to study the structure of that hole and optionally assess whether it is a round object or an object with a narrow-elongated shape, we must study the next element in the Taylor series, which is the second derivatives element. In some embodiments, the same logic is applied for a light object on a dark background (“hill”), however, in this case we are in an area of a local maximum point and not a local minimum.

According to some exemplary embodiments, to study the second element in the Taylor series, the H matrix is studied. Optionally, a second derivatives matrix, called a Hessian matrix is studied. In some embodiments, the matrix is used to identify blood vessels, for example long and narrow objects. Additionally or alternatively, the matrix is used to identify blobs that characterize a malignant tumor.

In some embodiments, in order to study the H matrix, the second derivatives is calculated for each examined point in the image. In some embodiments, Frangi proposes substituting the derivative action with a convolution of the picture with a Gaussian derivative. Optionally, the second derivatives of the image is calculated using a convolution with Gaussian derivatives in the appropriate directions.

According to some exemplary embodiments, the LOG operator combines a Laplacian calculation action (sum of second derivatives) with Gaussian smoothing, and it reflects the fact that smoothing of a derivative picture is replaced by a derivative of a smoothed picture. In some embodiments, the algorithms are used with the second derivatives separately and not with their sum, but the principle is the same.

According to some exemplary embodiments, the following characteristic of the convolution action is used:

$$\frac{\partial }{\partial {x}_{i}} ( {f}_{1} * {f}_{2} ) = \frac{\partial {f}_{1}}{\partial {x}_{i}} * {f}_{2} = {f}_{1} * \frac{\partial {f}_{2}}{\partial {x}_{i}}$$

In some embodiments, the picture is marked as: I(x,y) and the two-dimensional Gaussian with the studied point in its center, with a standard deviation σ G(x, y, σ)

In some embodiments, the equation is written as:

$$\frac{\partial }{\partial x} ( I * G ) = I * \frac{\partial G}{\partial x}$$

Optionally, the other derivatives are obtained in a similar manner.

According to some exemplary embodiments, substituting the derivative action with convolution with a Gaussian derivative is in fact calculating the derivatives on a picture that was previously smoothed with Gaussian at width σ. In some embodiments, the reason for conducting the differentiation on the smoothed picture instead of on the original picture is that in the original picture the blood vessels (the object we want to identify) is at least a few pixels wide, and therefore when we stand on a pixel in the blood vessel we will not identify a clear minimum point (there are no high gradients).

According to some exemplary embodiments, when the picture is smoothed with Gaussian whose width is a size order of the width of the blood vessel, the area outside the blood vessel will be blurred into the vessel. Therefore, even in the window around pixels in the middle of the blood vessel a clear power gradient in the direction of both ends of the blood vessel is obtained.

In some embodiments, if the blood vessel is assumed to be darker than the area outside it, then in the window around the pixel found in the middle of the blood vessel, the central pixel is at a clear minimum point of the power. Additionally, the power is increased in both directions along the axis perpendicular to the blood vessel axis. In some embodiments, by applying the above for this pixel a high second derivative in the relevant direction is achieved.

In some embodiments, when we move away from the center of the blood vessel we leave the minimum point, since studying the second derivatives enables us to approximately identify the midline of the blood vessel. This is achieved, as previously described using Gaussian smoothing, which makes it possible to refer to the blood vessel as a gradual slope whose center is in the minimum point. Optionally, the same method is used to discover objects that are not as long and narrow as blood vessels.

In some embodiments, if the object is round or square, for example, whose gray value is lower than its surroundings, then Gaussian smoothing is used to create a minimum point in the middle of the object with high second derivatives on both axes and not only on one axis, like in the case of the blood vessel.

According to some exemplary embodiments, the collection of second (smoothed) derivatives is used to produce the information about the blood vessel. In some embodiments, the H matrix containing the second derivatives for all directions is calculated.

In some embodiments, if the blood vessel flows parallel to the x axis, the first derivative in direction x will be very small, while the first derivative in direction y (perpendicular to the blood vessel axis) will be high. In some embodiments, an additional derivative in direction x yields a low result and an additional derivative in direction y yields a high result. In other words, we discover two important facts:
A. Derivative f_{yy} is much higher than fₓₓ
B. The mixed derivatives are low.

In some embodiments, based on the abovementioned facts, when the blood vessel is parallel to one of the axes—the H matrix is diagonal. Therefore, when turning the H matrix sideways, the blood vessel is turned to be parallel to one of the axes. In such a situation, the difference between fₓₓ and f_{yy} is the most prominent (the first fact above), which is how it is possible to identify whether it is a long and narrow object like a blood vessel. Moreover, we can also calculate the invert matrix required to turn H and thus to obtain the direction of the blood vessel.

According to some exemplary embodiments, when calculating the H matrix and turning it, the values of fₓₓ and f_{yy} in the turned matrix are the eigenvalues of the matrix. In some embodiments, if we place the λ₁ eigenvalues in increasing order, so that is the smallest eigenvalue and λ₂ the largest eigenvalue, then λ₁ is the eigenvalue that is compatible with the blood vessel axis. In some embodiments, based on Frangi article, when there is a significant difference between the values of λ₁ and λ₂ (the first is low and the second high)—the object is tubular, and when there is no significant difference between two eigenvalues (both are high)—the object is blob-like. A blood vessel is an example of a tubular object.

According to some exemplary embodiments, a new parameter R_{B} is defined to describe to what extent the object is blob-like (or tubular):

$${R}_{B} = \vert \frac{{λ}_{1}}{{λ}_{2}} \vert$$

Optionally, the more tubular the object, the lower the value for this parameter.

According to some exemplary embodiments, another parameter S is defined, whose shape is:

S=\sqrt{λ₁²+λ₂²}

In some embodiments, the eigenvalues express the intensity of the second derivative, then S will be higher if the second derivative is high (with a blood vessel, most of the contribution is from the direction perpendicular to the blood vessel axis).

Optionally, a high second derivative attests that we are near the minimum point (because if you move away toward the wall of the blood vessel, we are up a smoothed gradient, which is at a fixed value and therefore the second derivative is small). In some embodiments, a high second derivative filters noise (dark “cracks” in the picture that are not real blood vessels) optionally because small power differences create small gradients and therefore also small second derivatives, but this filtering is partial and the parameter is still sensitive to noise. Therefore, the main role of S is to ensure that we are in the center of the blood vessel (in the minimum zone).

According to some exemplary embodiments, an index which expresses the degree of similarity of a measured pixel to part of a blood vessel is defined. In some embodiments, The index is termed vesselness and is used to emphasize the blood vessel in the picture:

$$V ( x , y , σ ) = \{ \begin{matrix}0 & {λ}_{2} < 0 \\ exp ( - \frac{{R}_{B}^{2}}{ 2 {β}^{2}} ) \cdot ( 1 - exp ( - \frac{{S}^{2}}{2 {c}^{2}} ) ) & else\end{matrix}$$

Optionally, β and c are fixed when the sensitivity of the filter is controlled.

In some embodiments, the reason for resetting V when λ₂ is negative is because λ₂<0 when the tubular object is lighter than its surroundings, and this is not the case with blood vessels. In some embodiments, the value of the parameter V increases, when the value of R_{B} gets smaller (state of a tubular object) and when the value of S gets larger (we are near the center of the object). Optionally, multiplying the two factors by V creates AND conditions such that the parameter V is large when the two factors comprising it are simultaneously large. In some embodiments, if only one of the factors values is large and the second value small, the value of V will not be large.

In some embodiments, V is dependent on the width σ of the Gaussian (because the second derivatives at H are dependent on it). Therefore, recall that the calculation of the parameters that create V needs to be made for a series of σ values (that are compatible with the width of a blood vessel). The best values obtained are selected.

According to some exemplary embodiments, a map of V's values is presented in the entire image, or alternatively, to define a threshold value of V and obtain a binary image that is meant to mainly display the tumor (hot blobs) or vessel.

In some embodiments, using the Frangi filter (Multiscale vessel enhancement filtering Alejandro F. Frangi, Wiro J. Niessen, Koen L. Vincken, Max A. Viergever), the tumor and/or the blood vessel network of the tumor are highlighted. In some embodiments, the filter generates an image of hot blobs (tumor) and/or (hot low diameter tube) vessel. Optionally, using an interpolation algorithm, the Frangi image detection (tubes and/or blobs) is controlled. In some embodiments, enlarging the image by an interpolation algorithm enable the detection of blood vessels who are thinner than the tumor itself.

According to some exemplary embodiments, applying the Frangi filter on the cropped tumor area produces a filtered image of the tumor and/or vasculature. In some embodiments, the cropped tumor image is processed by the Frangi filter during radiotherapy, for example to monitor changes in heat generation of the tumor and vasculature. All other images are multiplied by this factor. In some embodiments, if the temperature of the tumor area is reduced during radiotherapy, then the image looks darker than baseline.

### Feature Extraction, and Quantitative Measure of Treatment Efficacy Generation

According to some exemplary embodiments, in the feature extraction stage, for example feature extraction 1006 entropy is calculated. In some embodiments, the concertation of blood vessel or tumor affect the homogeneity of the image, for example the higher concentration of blood vessel or tumor the lower homogeneity. Since in some embodiments entropy characterizes the homogeneity of the image, entropy is measured to evaluate the changes in vasculature and/or tumor over time or compared to a baseline.

According to some exemplary embodiments, once the images are processes entropy change from baseline is calculated using the following equation:

$$entropy change \% = \frac{\begin{matrix}( entropy of image after ( n ) Gy ) - \\ entropy of image before raditherapy\end{matrix}}{entropy of image before {raditherapy}_{baseline}} \times 100$$

In some embodiments, the entropy change value is a quantitative measure for the reduction in tumor size tumor or vasculature during radiotherapy.

### Validation Experiments

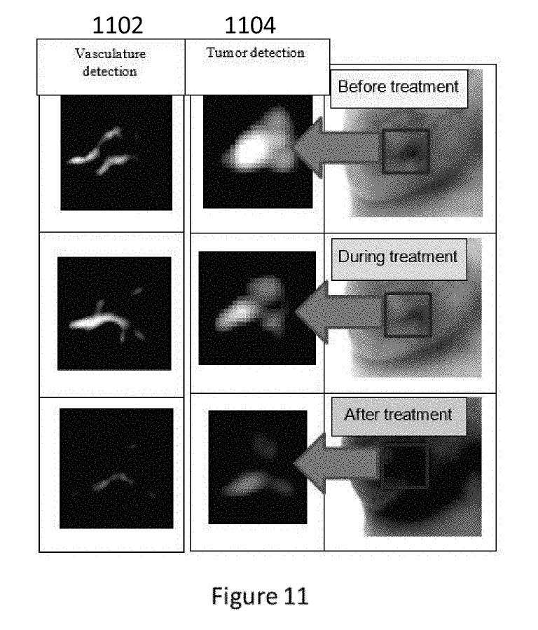

Reference is now made to FIG. 11 depicting reduction in tumor and vasculature signals during radiotherapy, according to some embodiments of the invention.

According to some exemplary embodiments, for example as shown in FIG. 11, thermal imaging of a cancer patient before, during (21 Gy), and at the end of treatment (39 Gy) reveal a marked decrease in detected vasculature 1102 and tumor 1104 during radiotherapy. The left-hand panel shows the tumor area, highlighted by the red box in the main image, after image processing vessel filtering. In the middle panel shows the tumor area, highlighted by the red box in the main image, after image processing tumor filtering, the “hot blobs” with high gradient of temperature (tumor). On the thermal image at the top, taken prior to the beginning of treatment, after image processing, the tumor and the concentration of blood vessels with malignant properties is apparent. In the middle thermal image, taken during radiotherapy (after 21 Gy), the vasculature and tumor is visibly reduced. In the thermal image at the bottom, taken at the end of treatment (after 39 Gy), a sharp decrease in the concentration of blood vessels and tumor is evident.

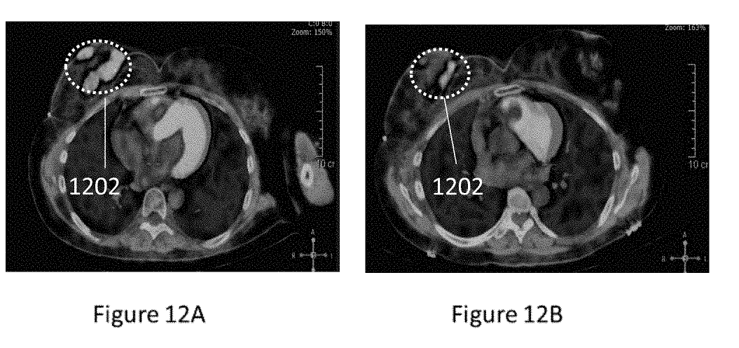

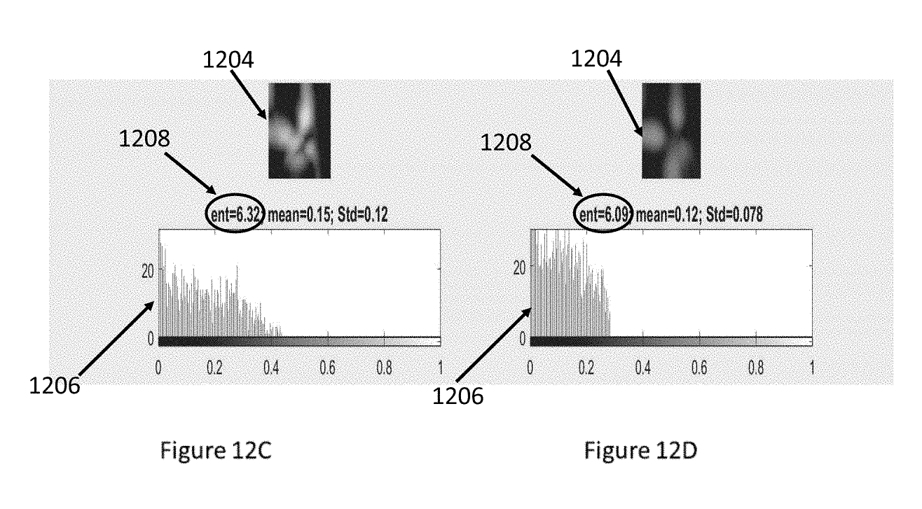

According to some exemplary embodiments, for example as shown in FIG. 12A, a tumor is visualized in ROI 1202 before radiotherapy treatment, using an imaging technique for example a PET-CT scan. In some embodiments, following radiotherapy, for example as shown in FIG. 12B, a reduction in the malignancy of the tumor in ROI 1202 is detected.

Reference is now made to FIGS. 12C and 12D depicting histograms of a cropped tumor area from thermal images before (12C) and after (12D) radiotherapy, according to some embodiments of the invention. According to some exemplary embodiments, for example as shown in FIGS. 12C and 12D, there is a reduction in the detected vasculature 1204 signal after radiotherapy in the processed images. In some embodiments, the reduction is also evident from histogram 1206. Additionally, a decrease is entropy values 1208 after radiotherapy is calculated using the algorithm discussed herein.

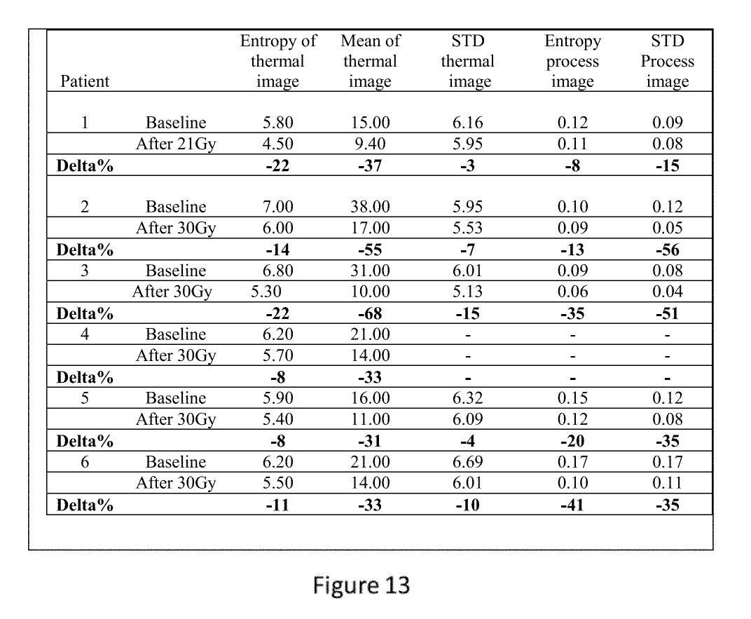

Reference is now made to FIG. 13 depicting a summarizing table for entropy values calculated before and after radiotherapy using the algorithm, according to some embodiments of the invention. According to some exemplary embodiments, a decrease in entropy in both the thermal image and the processed image was calculated for patients 1-6 after radiotherapy. This reduction in entropy values following radiotherapy is statistically significant (P=0.043). Additionally, the table in FIG. 13 summarizes the clinical outcomes of patients 1-6. The clinical outcomes are assessed by physicians according to a scale from 1 to 5: Grade 1: no improvement; Grade 2: slight decrease in tumor mass; Grade 3: moderate decrease in tumor mass; Grade 4: considerable decrease in tumor mass; 5: extreme decrease in tumor mass. Patients 7-14, who underwent radiotherapy as adjuvant treatment, were clinically disease free (CDF). The Spearman's Rho correlation showed a statistically significant correlation between the reduction in vasculature and clinical outcome R=0.89 P=0.04.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A method of monitoring a malignant tissue response to cancer treatment, comprising:
treating a malignant tissue by a cancer treatment;
acquiring throughout a radiotherapy treatment session, two or more thermal images of the treated malignant tissue;
processing said two or more thermal images to detect changes in said malignant tissue during said radiotherapy treatment; and
analyzing the processed images to determine an effect of said cancer treatment on said malignant tissue based on said detected changes.

2. The method according to claim 1, wherein said malignant tissue comprises vasculature and/or tumor.

3. The method according to claim 2, wherein said vasculature is located outside said tumor and/or within said tumor.

4. The method according to claim 1, further comprising delivering an indication to a user based if said effect is not a desired effect.

5. The method according to claim 2, wherein at least two thermal images are acquired and wherein said processing comprises comparing said at least two thermal images to determine one or more changes in said vasculature and/or said tumor that are indicative of a response of said malignant tissue to said cancer treatment.

6. The method according to claim 1, wherein said processing comprises identifying one or more of vessel irregularities associated with the presence of a tumor in said malignant tissue.

7. The method according to claim 2, wherein said detected vasculature comprises blood vessels supplying blood to said tumor.

8. The method according to claim 5, wherein said changes in vasculature comprise one or more of a change in vessel curvature, a change in vessel diameter, and a change in vascular density.

9. The method according to claim 2, wherein said processing comprises distinguishing between temperatures caused by inflammation of the tissue, temperatures associated with a change in the tumor, and temperatures associated with said vasculature.

10. The method according to claim 1, wherein said processing comprises applying one or more image processing algorithms configured to accentuate vasculature in the processed image.

11. The method according to claim 1, comprising:
detecting inflammation in said malignant tissue based on said processed images.

12. The method according to claim 1, wherein said cancer treatment comprises radiotherapy and/or brachytherapy and/or immunotherapy and/or hormonal treatment.

13. The method according to claim 1, wherein said cancer treatment comprises chemotherapy.

14. The method according to claim 1, wherein said acquiring is performed internally to the patient's body.

15. A system for monitoring cancer treatment using thermography, comprising:
a thermal imaging camera suitable for acquiring thermal images of a tissue region in which malignant tissue is present;
a controller programmed to operate said camera two or more times throughout a radiotherapy treatment session according to one or more predefined protocols;
memory circuitry for storing one or more thermal images and/or processed images; and
a processor configured to analyze the acquired thermal images for indicating the tissue response to a cancer treatment based on a condition of vasculature associated with said malignant tissue; wherein said processor compares said acquired thermal images to said stored thermal images and/or said stored processed thermal images.

16. The system according to claim 15, wherein said processor is programmed to apply one or more image processing algorithms designed to identify said vasculature condition or changes therein.

17. The system according to claim 15, wherein said system is configured to provide a progress related indication for determining the efficacy of said cancer treatment.

18. The system according to claim 15, wherein said system is configured to be integrated in and/or added onto an irradiating modality.

19. The system according to claim 15, wherein said cancer treatment comprises radiotherapy and/or chemotherapy and/or brachytherapy and/or immunotherapy.

20. The system according to claim 15, wherein said thermal imaging camera is shaped and sized to be inserted through a body orifice.
